(19) United States
(12) Patent Application Publication
(10) Pub. No.: US 2025/0064982 A1
(43) Pub. Date: Feb. 27, 2025

(54) RECOMBINANT AAV VECTORS FOR TREATING GLUTARIC ACIDURIA TYPE I

(71) Applicant: SHANGHAI VITALGEN BIOPHARMA CO., LTD., Shanghai (CN)

(72) Inventors: Lu GUO, Beijing (CN); Xi ZHU, Shanghai (CN); Shin-Shay TIAN, Shanghai (CN); Xiaoping ZHAO, Shanghai (CN)

(73) Assignee: SHANGHAI VITALGEN BIOPHARMA CO., LTD., Shanghai (CN)

(21) Appl. No.: 18/948,694

(22) Filed: Nov. 15, 2024

Related U.S. Application Data
(63) Continuation of application No. PCT/CN2023/094298, filed on May 15, 2023.

(30) Foreign Application Priority Data
May 16, 2022 (WO) PCT/CN2022/093084

(51) Int. Cl. A61K 48/00; C12N 9/02; C12N 15/86

(52) U.S. Cl. CPC A61K 48/0058; C12N 9/001; C12N 15/86; C12Y 103/08006; C12N 2750/14122; C12N 2750/14143

(57) ABSTRACT

The present disclosure relates to codon-optimized sequences coding for hGCDH polypeptide and recombinant adeno-associated virus (rAAV) vectors comprising one of said sequences under the control of a promoter component. Also provided herein are viral particles comprising the rAAV vector, a pharmaceutical composition comprising the rAAV vector or the viral particles, and uses thereof in treating Glutaric aciduria type I (GA-I).

### CROSS-REFERENCE

This application is a continuation application of PCT International Application No. PCT/CN2023/094298, filed May 15, 2023, which claims the benefit of China Patent Application PCT/CN2022/093084, filed May 16, 2022, which all is incorporated herein by reference in its entirety.

### FIELD OF THE INVENTION

The present disclosure relates to the technical field of gene therapy. Specifically, the present disclosure provides a recombinant adeno-associated viral (rAAV) vector comprising a nucleotide sequence encoding human glutaryl-coA dehydrogenase (GCDH), for treating a disorder or condition caused by the deficiency of glutaryl-coA dehydrogenase (GCDH), especially Glutaric aciduria type I (GA-I).

### SEQUENCE LISTING

In accordance with 37 CFR § 1.52(e)(5) and with 37 CFR § 1.831, the specification makes reference to a Sequence Listing submitted electronically as a .xml file named 20241115_19684.0089FPWO_sql.xml. Said .XML copy is 66,000 bytes in size. The entire contents of the Sequence Listing are hereby incorporated by reference.

### BACKGROUND

First described in 1974, glutaric aciduria is an inherited neurometabolic disorder [1]. With elevated glutaric acid (GA) in plasma, urine and cerebrospinal fluid (CSF), patients suffer from neurodegenerative disorder [1,2]. Glutaric aciduria type I (GA-I) is an autosomal recessive metabolic disorder caused by the deficiency of glutaryl-CoA dehydrogenase (GCDH; EC 1.3.99.7) [3]. Located in mitochondria, GCDH is a key enzyme in the metabolism of L-lysine, L-hydroxylysine and L-tryptophan. Glutaryl-CoA is an intermediate of this pathway and is dehydrogenated and decarboxylated to crotonyl-CoA by GCDH. When GCDH is deficient, glutaryl-CoA cannot be catalyzed correctly and the by-product—GA is generated. The accumulation of GA in the brain causes neurotoxicity and neurodegenerative disorder [4]. Patients will suffer from macrocephaly, hypotonia and acute encephalopathy crisis. Without proper treatment, GA-I patients' life expectancy could be only 2-3 years. In mammals, GA can bind to carnitine and forms glutarylcarnitine (C5DC), so that GA is eliminated and detoxicated to some extent. In clinic, carnitine supplementation is a widely used treatment for GA-I patients. Carnitine supplementation not only decreases GA levels but also prevents secondary carnitine deficiency [5].

The global incidence of GA-I is estimated to be 1/100,000. About 75,000 patients suffer from this inherited metabolic disorder. Current treatments include diet control and carnitine supplementation. However, diet control of protein restriction could be hard to adhere in daily life, and carnitine supplementation cannot eliminate 3-hydroxy glutaric acid (3-OH-GA) and glutaconic acid [6].

There still exists an unmet need for an efficient treatment of disorders caused by the deficiency of GCDH, especially a curative treatment for GA-I.

### SUMMARY OF THE INVENTION

Gene therapy has been proven to be efficient in treating inherited metabolic disorders, as demonstrated in both animal models and clinical trials. Gene replacement strategy based on adeno-associated virus (AAV) has been proven to be effective in a variety of recessive genetic disorders.

GA-I is a neurometabolic disorder and GCDH deficiency mainly causes damages to the central nervous system (CNS). Evidences have suggested that blood-brain barrier has low permeability for dicarboxylic acid, GCDH deficiency in the CNS cells causes in situ GA accumulation and therefore neurotoxicity [7]. Normalization of the CNS amino acid metabolism and decrease of the CNS GA accumulation by delivering rAAV carrying GCDH expression cassette directly to the CNS could benefit GA-1 patients. The Gcdh^{-/-} mice have similar life expectancy as the wild-type C57BL/6 mice [8]. To mimic the situation of acute encephalopathic crisis, GCDH knockout mice were challenged with high protein diet [9]. High protein diet (HPD) challenge was lethal to the 4-week-old Gcdh^{-/-} mice within 2-3 days. Under HPD challenge, Gcdh^{-/-} mice developed GA accumulation, vasogenic oedema, neuronal loss, paralysis, seizures [9]. Thus, Gcdh^{-/-} mice exposed to high protein may be a useful model of human GA-1 including developmentally dependent striatal vulnerability [9].

For the first time, the present inventors have developed an rAAV vector comprising an optimized GCDH coding sequence under the control of a specially designed promoter, and verified its effect in alleviating symptoms due to GA accumulation in Gcdh^{-/-} mouse model under HPD challenge, thus completing the invention.

Therefore, in a first aspect, the present application provides an isolated nucleic acid molecule, comprising a nucleotide sequence selected from a group of nucleotide sequences consisting of SEQ ID NOs: 11-18 (coding sequences C1-C8), wherein the nucleotide sequence encodes human GCDH polypeptide having an amino acid sequence as shown in SEQ ID NO: 33. In a specific embodiment, the isolated nucleic acid molecule, comprising a nucleotide sequence as shown in SEQ ID NO: 12 (coding sequence C2). The coding sequence of the present application has a reduced CpG number as compared to the wild-type coding sequence of hGCDH as shown in SEQ ID NO: 10.

In a second aspect, the present application provides a promoter component having a nucleotide sequence selected from a group of nucleotide sequences consisting of SEQ ID NOs: 2-9 (promoter components P1-P8). In a preferred embodiment, the promoter component has a nucleotide sequence as shown in SEQ ID NO: 5 or SEQ ID NO: 9 (P4 or P8).

In a third aspect, the present application provides an expression cassette, comprising a coding sequence for the human GCDH polypeptide having an amino acid sequence as shown in SEQ ID NO: 33, operatively linked to a promoter component, wherein the coding sequence has a nucleotide sequence selected from a group of nucleotide sequences consisting of SEQ ID NOs: 11-18, and the promoter component has a nucleotide sequence selected from a group of nucleotide sequences consisting of SEQ ID NOs: 2-9. In a preferred embodiment, the expression cassette comprising a coding sequence having a nucleotide sequence as shown in SEQ ID NO: 12, under the control of a promoter component having a nucleotide sequence as shown in SEQ ID NO: 5 or SEQ ID NO: 9. In a more specific embodiment, the expression cassette comprises a nucleotide sequence as shown in SEQ ID NO: 20 or SEQ ID NO: 21 (V2 or V3).

In a fourth aspect, the present application provides a rAAV vector, comprising the isolated nucleic acid molecule of the first aspect or the expression cassette of the third aspect. In a preferred embodiment, the rAAV vector provides a desirable expression level of human GCDH protein in target tissues, e.g., disease relevant tissues in the CNS.

In a fifth aspect, the present application provides an AAV viral particle comprising the rAAV vector packaged into an AAV capsid. The AAV capsid can be derived from any AAV serotype, e.g., AAV1, AAV2, AAV3B, AAV5, AAV6, AAV7, AAV8, AAV9, AAVLK03, AAVS3, AAVKP1, AAVrh10, AAVNP40, AAVNP59, AAV-DJ, AAVAnc80L65, AAVsL65, AAVHSC15, AAVC102, AAV204, AAV214. In one embodiment, the AAV capsid is a capsid with CNS tropism, such as AAV9 or AAV PHP.B capsid.

In a sixth aspect, the present application provides a pharmaceutical composition comprising the rAAV vectors of the fourth aspect or the viral particle of the fifth aspect, and a pharmaceutically acceptable excipient.

In a seventh aspect, the present application provides a method for treating GA-I in a subject in need thereof, comprising administering to the subject a therapeutically effective amount of the rAAV vector of the fourth aspect, the rAAV particle of the fifth aspect or the pharmaceutical composition of the sixth aspect.

In an eighth aspect, the present application provides use of the rAAV vector of the fourth aspect, the rAAV particle of the fifth aspect or the pharmaceutical composition of the sixth aspect in treating GA-I patients.

### BRIEF DESCRIPTION OF THE DRAWINGS

FIG. 1 shows the illustration of GCDH expression vectors in Example 1.

FIG. 2 shows the GCDH protein expression comparison of P0-P8 in U87 MG cells.

FIG. 3 shows the GCDH protein expression comparison of P0-P8 in HEK293 cells.

FIG. 4 shows the illustration of GCDH CDS evaluation vectors in Example 2.

FIG. 5 shows the GCDH protein expression evaluation of C0-C8 in U87 MG cells.

FIG. 6 shows the GCDH protein expression evaluation of V1-V3 in U87 MG cells.

FIG. 7 shows the GCDH enzyme activity evaluation of V1-V3 in SH-sy5y cells.

FIG. 8 shows the survival curves of the Gcdh^{-/-} mice after administration of rAAV9-V1 under high protein diet challenge.

FIG. 9 shows the survival curves of the Gcdh^{-/-} mice after administration of rAAV9-V2 under high protein diet challenge.

FIG. 10 shows the GCDH protein expression evaluation of rAAV9-V1 and rAAV9-V2 in Gcdh^{-/-} mice.

FIG. 11 shows the LC-MS analysis of GA levels in the brain, liver and plasma of the survived and dead Gcdh^{-/-} mice after rAAV9-V2 administration.

FIG. 12 shows the results of LC-MS/MS analysis of GA and 3-OHGA levels in different tissues (cerebrospinal fluid (only for GA), brain, liver, serum, and urine) at 4 weeks after AAV administration.

FIG. 13 shows the results of LC-MS/MS analysis of GA and 3-OHGA levels in different tissues (cerebrospinal fluid (only for GA), brain, liver, serum, and urine) at 13 weeks after AAV administration.

FIG. 14 shows the GCDH protein levels in different tissues of mice 4 weeks and 13 weeks after administering with a high dose (1.0×10¹⁰ vg) of rAAV9-V2.

FIG. 15 shows the HE staining of mouse brain 13 weeks after administration of AAV or vehicle (200X).

### DETAILED DESCRIPTION OF THE INVENTION

Unless specifically defined elsewhere in this document, all of the technical and scientific terms used herein have the meaning commonly understood by one of ordinary skill in the art to which this invention belongs.

As used herein, including the appended claims, the singular forms of words such as “a”, “an”, and “the”, include their corresponding plural references unless the context clearly dictates otherwise.

In the context of the present disclosure, unless being otherwise indicated, the wording “comprise”, and variations thereof such as “comprises” and “comprising” will be understood to imply the inclusion of a stated element, e.g. an amino acid sequence, a nucleotide sequence, a property, a step or a group thereof, but not the exclusion of any other elements, e.g. amino acid sequences, nucleotide sequences, properties and steps. When used herein the term “comprise” or any variation thereof can be substituted with the term “contain”, “include” or sometimes “have” or equivalent variation thereof. In certain embodiments, the wording “comprise” also include the scenario of “consisting of”.

Coding Sequence of hGCDH

As an essential part of the expression cassette, the present disclosure first provides a group of codon-optimized nucleotide sequences encoding for hGCDH polypeptide having an amino acid sequence as shown in SEQ ID NO: 33.

By “isolated nucleic acid”, it means a DNA or RNA which is removed from all or a portion of a polynucleotide in which the isolated polynucleotide is found in nature, or is linked to a polynucleotide to which it is not linked in nature. An isolated nucleic acid molecule “comprising” a specific nucleotide sequence may include, in addition to the specified sequence, operably linked regulatory sequences that control expression of the coding region of the recited nucleic acid sequences. Due to the codon degeneracy, one skilled in the art understands that any specific amino acid sequence can be coded by several different nucleotide sequences.

“Codon-optimized coding sequence” herein refers to a nucleotide sequence coding for hGCDH protein modified from their wild-type coding sequence accommodating codon bias. Optimization may be achieved by reducing sequence complexity, adjusting GC content, adjusting codon usage and/or avoiding rare codons. The coding sequence which has been codon optimized usually shows an increased translational efficiency of the gene of interest (GOI), leading to a higher protein expression.

The codon-optimized coding sequence of hGCDH of the present application has a reduced CpG number as compared to the wild-type coding sequence of hGCDH as shown in SEQ ID NO: 10. “CpG content” or “CpG number” refers to the content or numbers of cytosine (C) guanine (G) dinucleotides linked by phosphate (p) in a DNA sequence. “CpG islands” are genomic regions where CpG dinucleotides occur with a higher frequency. For example, the algorithm described by Gardiner-Garden and Frommer (1987) can be used to determine the presence of CpG islands. Specifically, a region containing at least 200 bp, in which the proportion of GCs exceeds 50%, and the observed/predicted ratio of CpG is higher than 0.6, this region is called “CpG island”. The predicted value of CpG can be calculated as the number of Cs in an observation window multiplied by the number of Gs in the window, divided by the window length. In mammals, unmethylated CpGs of exogenous genes are recognized by TLR9 resulting in activation of CD8⁺ T cells to clear the infected cells, which is not favored for long-term expression of the exogenous genes. Therefore, in order to express the GCDH-encoding gene more efficiently, it is preferable to reduce the numbers of CpGs in the hGCDH coding sequences. The coding sequences of the present invention preferably have lower CpG contents. When the CpG content is a factor to consider during codon optimization, it further increases the complexity of sequence design and validation work.

The wild-type coding sequence of hGCDH as shown in SEQ ID NO: 10 has a CpG number of 73. Preferably, the hGCDH coding sequence of the present application has a CpG number lower than 73. For example, the hGCDH coding sequence of the present application has a CpG number no more than 65, no more than 55, no more than 50, no more than 45, no more than 40, no more than 35, no more than 30, no more than 25, no more than 20. For example, the hGCDH coding sequence of the present application has a CpG number at least 10% less than the CpG number of the wild-type coding sequence of hGCDH (e.g., the nucleotide sequence as shown in SEQ ID NO: 10), preferably at least 20% less, at least 30% less, at least 40% less, at least 50% less, at least 60% less, at least 70% less, at least 80% less, at least 90% less, than the CpG number of the wild-type coding sequence of hGCDH (e.g., the nucleotide sequence as shown in SEQ ID NO: 10). In a specific embodiment of the present application, the hGCDH coding sequence having a nucleotide sequence of SEQ ID NO: 12 (coding sequence C2) has a CpG number as low as 16.

In preferred embodiments, the codon-optimized coding sequence for human GCDH protein comprises or consists of a nucleotide sequence selected from SEQ ID No: 11-18. In one particularly preferred embodiment, the codon-optimized coding sequence for human GCDH protein comprises or consists of a nucleotide sequence as shown in SEQ ID No: 12.

### Regulatory Sequences

Further, an expression cassette can comprise one or more regulatory sequences in addition to the coding sequence. Regulatory sequence can be selected from one or more of promoter, enhancer, polyadenylation sequence, and translation termination signal. A certain combination of regulatory sequences of the present disclosure can achieve unexpected effect in improving the expression efficiency of the coding sequence.

In one aspect, the present application provides a series of new promoter components. By “promoter component”, it refers to a sequence component located 5′ upstream of the coding sequence, and is consisted of a promoter and optionally additional element(s) such as enhancer and/or an intron-derived fragment, in which the enhancer usually locates upstream of the promoter and the intron-derived fragment usually locates downstream of the promoter.

The term “promoter” refers to a DNA sequence enables initiation of transcription of a downstream gene under the control of the said promoter. Promoters include but not limited to constitutive promoters, cell type-specific promoters, tissue-specific promoters, development stage-specific promoters. Promoter can be a naturally occurring promoter of a gene, a modified version of a naturally occurring promoter or a synthetic promoter.

In preferred embodiments, the promoter of the present disclosure can be a constitutive promoter. For example, the promoter can be a cytomegalovirus (CMV) promoter, a chicken β-actin (CBA) promoter, or a human elongation factor-1 alpha (EF-1α) promoter. In one embodiment, the promoter is a CMV promoter having the nucleotide sequence as shown in SEQ ID NO: 25. In one embodiment, the promoter is a CBA promoter having the nucleotide sequence as shown in SEQ ID NO: 26. In one embodiment, the promoter is an EF-1α core promoter having the nucleotide sequence as shown in SEQ ID NO: 29.

“Enhancer” is a regulatory DNA sequence which can enhance the transcription of the GOI in AAV together with the promoter. In some embodiments, the promoter component of the present application comprises an enhancer. More preferably, the enhancer can be a CMV enhancer. For example, the CMV enhancer can have a nucleotide sequence as shown in SEQ ID NO: 24.

“Intron-derived fragment” is a sequence derived from an intron of a gene. It has been reported that gene transcription can be enhanced by a splicing-competent intron. In preferred embodiments, the promoter component or the expression cassette of the present application comprises an intron-derived fragment.

In some embodiments, the intron-derived fragment is originated from the intron of SV40, e.g., an intron-derived fragment having a nucleotide sequence as shown in SEQ ID NO: 28.

In some embodiments, the intron-derived fragment is originated from any intron of the human GCDH. For example, the intron sequence is consisted of one or more fragments derived from one or more intronic regions of the human GCDH gene. In preferred embodiments, the promoter component or the expression cassette of the present application comprises an intron-derived fragment having a nucleotide sequence as shown in any of SEQ ID NO: 27, 30, or 31 (hGCDH intron 1, hGCDH intron 2 or hGCDH intron 3, respectively). For example, the intron-derived fragment can be a combination of any two or three of hGCDH intron 1 (SEQ ID NO: 27), hGCDH intron 2 (SEQ ID NO: 30) and hGCDH intron 3 (SEQ ID NO: 31).

In some embodiments, the intron-derived fragment is a hybrid intron. By “hybrid intron”, it refers to an intron fragment comprising at least two sequences from different origins, or from the same origin but not in consecutive in natural state. For example, the promoter component or the expression cassette of the present application comprises a hybrid intron as the intron-derived fragment, wherein the hybrid intron comprises two intron-derived fragments originated from chicken β-actin (CBA) and minute virus of mice (MMV) introns as shown in SEQ ID NO: 32.

Preferably, the intron-derived fragment has a total length of about or less than 200 bp, about or less than 250 bp, about or less than 300 bp, about or less than 350 bp, about or less than 400 bp.

In some embodiments, the promoter component of the present application comprises a CMV enhancer, a CBA promoter, and optionally an intron-derived fragment. In this case, the intron-derived fragment is preferably an intron derived from the hGCDH gene, or SV40 (e.g., SEQ ID NO: 28). In specific embodiments, the promoter component comprises or consists of a nucleotide sequence as shown in any one of SEQ ID NOs: 2-4.

In some embodiments, the promoter component of the present application comprises an EF-1α promoter, e.g., an EF-1α core promoter (e.g., SEQ ID NO: 29), and an intron-derived fragment. In this case, the intron-derived fragment is preferably an intron derived from the hGCDH gene, or a hybrid intron (e.g., SEQ ID NO: 32). In specific embodiments, the promoter component comprises or consists of a nucleotide sequence as shown in any one of SEQ ID NOs: 5-9. In a more preferred embodiment, the promoter component comprises or consists of a nucleotide sequence as shown in SEQ ID NO: 5 or 9.

In a preferred embodiment, the promoter component has a length of no more than 1,000 bp, no more than 900 bp, no more than 850 bp, no more than 800 bp, no more than 700 bp, no more than 600 bp, no more than 500 bp, or no more than 400 bp, due to the limited packaging capacity of AAV.

### Expression Cassette

The term “expression cassette” herein refers to a DNA component included in a vector (e.g., rAAV vector) and consisted of a gene (e.g., human GCDH gene) to be expressed in a host cell transfected by the vector and regulatory sequence(s).

By optimizing cDNA sequence (codon) of the human GCDH gene, and the regulatory sequences, in particular the promoter component, the expression cassette of hGCDH inserted into an AAV vector can provide a desirable expression level and a reduced immunogenicity after the rAAV is delivered into a subject.

In one specific embodiment, the expression cassette comprises any of the coding sequences having a nucleotide sequence as shown in any of SEQ ID NOs: 11-18, preferably a nucleotide sequence as shown in SEQ ID NO: 12, operatively linked to a promoter component having a nucleotide sequence as shown in any of SEQ ID NOs: 2-9, preferably a nucleotide sequence as shown in SEQ ID NOs: 5 or 9. By “operatively linked”, it means that the promoter component is in a functionally appropriate location and/or orientation in relation to the coding sequence so as to control the transcription of the coding sequence.

In specific embodiments, the expression cassette comprises or consists of a nucleotide sequence as shown in any one of SEQ ID NOs: 19-21, preferably as shown in SEQ ID NO: 20 or SEQ ID NO: 21.

### Recombinant AAV Vectors and Viral Particles

The nucleic acid molecule or the expression cassette of the present disclosure can be constructed into a recombinant AAV (rAAV) vector, to obtain rAAV particles for delivery into a subject in need thereof.

In addition to the inserted nucleotide sequence as described above, the rAAV vectors are in self-complementary form. The rAAV vector is comprised of two inverted terminal repeat (ITR) sequences at both ends of the inserted nucleotide sequence. The ITR of the present disclosure can be ITR derived from any AAV serotypes. When reference is made to serotype of AAV ITR, the phrase “derived from” means that the ITR can be the ITR of a certain serotype or a variant derived therefrom with modification(s). In a preferred embodiment of the present disclosure, the rAAV vector comprises two ITRs derived from AAV2. For example, the rAAV vector comprises two AAV2 ITRs, or comprises a wild-type AAV2 ITR and a truncated version of AAV2 ITR lacking the region C or region C′. For example, the wild-type AAV2 ITR locates at the 5′ of the inserted nucleotide sequence, while the AAV2 ITR variant locates at the 3′ of the inserted nucleotide sequence; or vice versa. In one embodiment, the ITRs comprised in the rAAV of the present application have nucleotide sequences as shown in SEQ ID NO: 22 (5′ ITR) and SEQ ID NO: 23 (3′ ITR).

The rAAV genome was packaged into an AAV capsid. The capsid can be derived from any AAV serotype known in the art or characterized in the future. The capsid and ITRs can be derived from the same serotype of AAV or from different serotypes of AAV. For example, the capsid can be a capsid suitable for intravenous (IV) delivery (e.g., IV injection) to the peripheral tissues. The term “peripheral tissue” in the context of the present application refers to any tissue that is not a part of the brain or spinal cord. For example, the capsid can be a capsid suitable for the nervous system delivery, e.g., intrathecal, intracisterna magna, or intracerebroventricular delivery (e.g., by injection). In some embodiments, the AAV vector comprises a capsid of AAV1, AAV2, AAV4, AAV5, AAV7, AAV8, AAV9, AAVrh10, AAV PHP.B, AAV2.7m8, or AAVAnc80L65 serotype, or a variant thereof. In a preferred embodiment, the capsid is the AAV9 capsid.

### Pharmaceutical Composition

The term “pharmaceutical composition” refers to a composition suitable for delivering to a subject. The pharmaceutical composition of the present disclosure comprises the isolated nucleic acid, the rAAV vector or the viral particle of the present disclosure and a pharmaceutically acceptable excipient. Conventional pharmaceutically acceptable excipients are known in the art and can be solid or liquid excipients. In one embodiment, the pharmaceutical composition can be a liquid for injection.

### Delivery

The terms “administration”, “administering”, “treating” and “treatment” as used herein, when applied to a subject, e.g., an animal, including human, or to cell, tissue, organ, or biological fluid, means contact of an exogenous pharmaceutical, therapeutic, diagnostic agent, or composition with the subject, cell, tissue, organ, or biological fluid. Treatment of a cell encompasses contact of a reagent with the cell, as well as contact of a reagent with a fluid, where the fluid is in contact with the cell. The term “administration” and “treatment” also include in vitro and ex vivo treatments, e.g., of a cell, by a reagent, diagnostic, binding compound, or by another cell.

In some embodiments, the rAAV vector of the present application can be administered into a subject via systemic delivery or local delivery. In some embodiments, the rAAV vector of the present application can be delivered to peripheral tissues or organs rather than into the nervous system, e.g., into peripheral blood, via any parental or enteral route. For example, the rAAV vector of the present application can be administered by intravenous (IV), intramuscular (IM), subcutaneous (SC), intra-arterial, intraperitoneal (IP), intradermal, transdermal, oral, nasal or rectal route. In some embodiments, the rAAV vector of the present application can be delivered into the nervous system, e.g., into cerebrospinal fluid (CSF). For example, the rAAV vector of the present application can be administered by intracerebroventricular (ICV), intrathecal, or intracisterna magna (ICM) route. For example, the rAAV can be delivered by injection. In some embodiment, the rAAV vector can be delivered by a combined administration via more than one delivering route. For example, the rAAV vector can be delivered to both the peripheral tissues and the nervous system, successively or simultaneously.

The rAAV vector can be administered via a single dose or multiple doses. In a specific embodiment, the rAAV vector is administered via a single injection.

### Therapeutic Uses

The term “treat”, “treating” or “treatment” includes to cure or at least to alleviate the symptoms of a disorder or condition caused by GCDH deficiency, such as the symptoms of GA-I.

### EXAMPLES

To facilitate the understanding and utilization of the present invention, the merits of the present invention will be described in more details with reference to examples and appended drawings. However, it should be understood that the following examples only intend to exemplify the present invention without any intention in limiting the scope of the present invention. The scope of the present invention should be defined by the claims.

### Example 1. A Constitutive Promoter with Minimum of CPG Number for GCDH Protein Expression

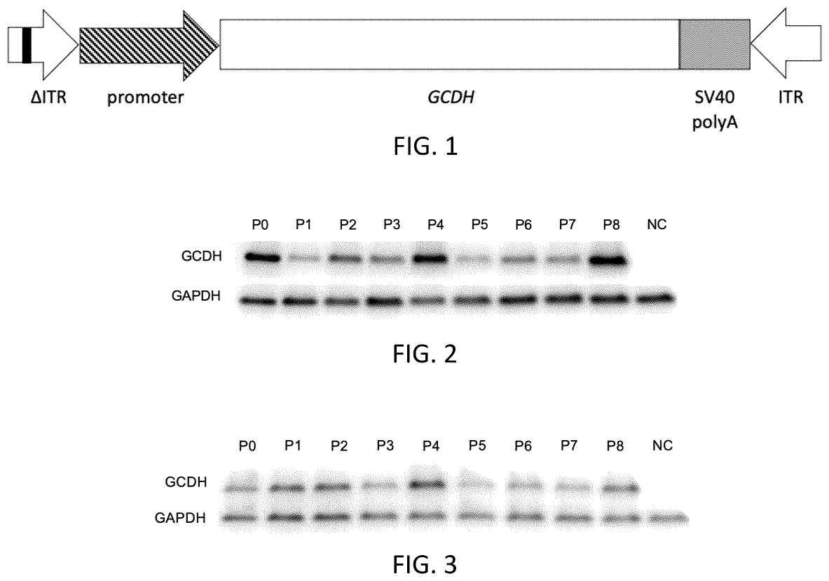

A GCDH expression AAV vector V1 was constructed and consists of ITRs, cmv enhancer/promoter (P0, SEQ ID NO: 1), wild type GCDH CDS and SV40 polyA. However, due to the silencing risk of CMV enhancer/promoter, the CMV enhancer/promoter was replaced by artificially synthesized promoter components P1-P8 (SEQ ID NOs: 2-9, respectively) to achieve more robust expression of GCDH. Each of the tested promoter components (P1-P8) includes an enhancer and/or an intron, in addition to a constitutive promoter. The specific structure of the promoter components P1-P8 are provided in Table 1. Illustration of the vector structure is shown in FIG. 1.

TABLE 1
Information of the promoter components P0-P8

| No. | |
|---|---|
| P0 | CMV enhancer (SEQ ID NO: 24)-CMV |
| | promoter (SEQ ID NO: 25) |
| P1 | CMV enhancer-chicken β-actin |
| | promoter (SEQ ID NO: 26) |
| P2 | CMV enhancer-chicken β-actin promoter-hGCDH |
| | intron 1 (SEQ ID NO: 27) |
| P3 | CMV enhancer-chicken β-actin promoter-SV40 |
| | intron (SEQ ID NO: 28) |
| P4 | EF-1α core promoter (SEQ ID NO: 29)-hGCDH intron 1 |
| P5 | EF-1α core promoter-hGCDH intron 2 (SEQ ID NO: 30) |
| P6 | EF-1α core promoter-hGCDH intron 3 (SEQ ID NO: 31) |
| P7 | EF-1α core promoter-hGCDH intron 1-hGCDH intron 2 |
| P8 | EF-1α core promoter-hybrid intron (SEQ ID NO: 32) |

U87 MG or HEK293 cells were maintained in DMEM+10% FBS and passaged every 3 days by TrypLE. The day before transfection, cells were inoculated to a 24-well plate in 1×10⁵/cm². Plasmids were transfected into U87 MG or HEK293 cells using Lipofectamine 3000 Transfection Reagent (Invitrogen, L3000008) following the user's guide. 72 hours after transfection, cells were collected in RIPA lysis buffer (Beyotime, P0013C) with protease inhibitor cocktail (Roche, 04693159001) and SDS-PAGE loading buffer (Cowin Bio, CW0027), denatured for 10 min at 95° C., centrifuged at 12,000 rpm for 10 min. Supernatants were separated in 4%-10% SDS-PAGE gel (Cowin Bio, CW0022M), and blotted onto 0.2 μm PVDF transfer membrane (Merck, ISEQ00010). The protein levels of GCDH and housekeeping gene GAPDH were detected by an antibody against GCDH (Abcam, ab232774) and GAPDH (Cell Signaling Technology, 21185), respectively. The Western Blot images are shown in FIG. 2 and FIG. 3.

The results as shown in FIG. 2 and FIG. 3 suggest that P4 and P8 mediated the highest GCDH protein expression in both U87 MG and HEK293 cells.

### Example 2. Codon Optimization to Minimize Immunogenicity Risks and Enhance Expression

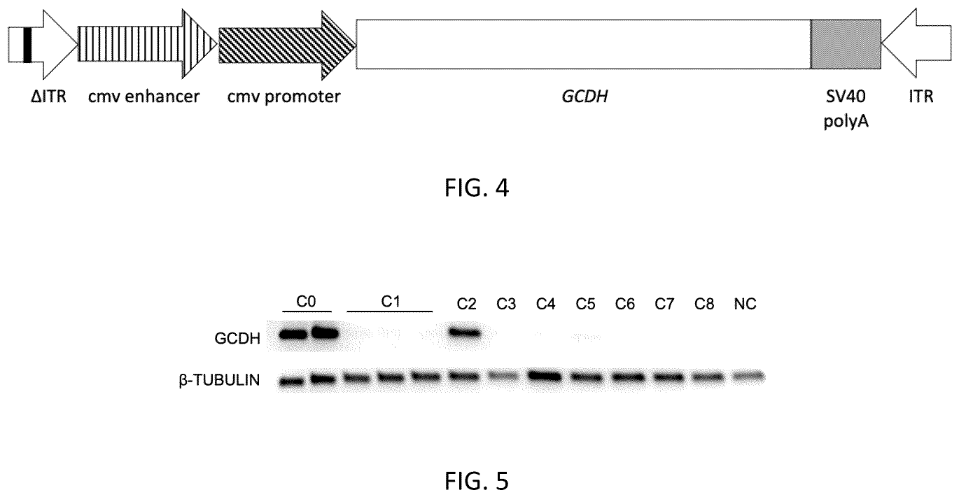

CpGs in AAV vectors have been reported to cause immunoreaction and exogenous gene silencing [10,11]. In this example, the coding sequence of GCDH was optimized to enhance expression and to reduce the numbers of CpGs, and the CMV promoter was used to test the expression efficiency of the codon optimized sequences. The vector structure is shown in FIG. 4. A total of eight different optimized coding sequences were synthesized, namely C1-C8, having nucleotide sequences as shown in SEQ ID NOs: 11-18. The CpG numbers of the modified coding sequences C1-C8 together with the wild-type coding sequence C0 are summarized in Table 2.

TABLE 2
CpG numbers of the codon optimized sequences C0-C8

| No. | CpG number | No. | CpG number | No. | CpG number |
|---|---|---|---|---|---|
| C0 | 73 | C3 | 30 | C6 | 46 |
| C1 | 51 | C4 | 70 | C7 | 33 |
| C2 | 16 | C5 | 65 | C8 | 46 |

To evaluate the expression of codon optimized sequences C1-C8, U87 MG cells were maintained in DMEM+10% FBS and passaged every 3 days by TrypLE. The day before transfection, cells were inoculated to 24-well plate in 1×10⁵/cm². Plasmids were transfected into U87 MG using Lipofectamine 3000 Transfection Reagent (Invitrogen, L3000008) following the user's guide. 72 hours after transfection, cells were collected in RIPA lysis buffer (Beyotime, P0013C) with protease inhibitor cocktail (Roche, 04693159001) and SDS-PAGE loading buffer (Cowin Bio, CW0027), denatured for 10 min at 95° C., centrifuged at 12,000 rpm for 10 min. Supernatants were separated in 4%-10% SDS-PAGE gel (Cowin Bio, CW0022M), and blotted onto 0.2 μm PVDF transfer membrane (Merck, ISEQ00010). The protein levels of GCDH and housekeeping gene GAPDH were detected by an antibody against GCDH (Abcam, ab232774) and β-TUBULIN (Proteintech, 66240-1), respectively. The Western Blot images are shown in FIG. 5.

The results as shown in FIG. 5 suggest that the codon optimized sequence C2 mediated the highest GCDH protein expression in the U87 MG cells.

### Example 3. GCDH Constructs Comprising Optimized Promoter Component and Coding Sequence

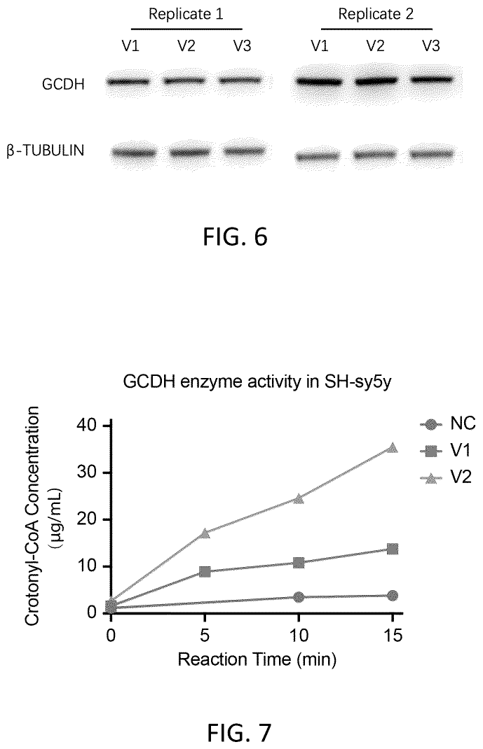

The codon optimized sequence C2 was identified in Example 2 as containing the best GCDH protein coding sequence. Therefore, C2 was combined with either of the top 2 promoter components identified in Example 1 (P4 and P8), resulting in two plasmid constructs V2 (P4-C2) and V3 (P8-C2) for further evaluation of GCDH protein expression efficiency. U87 MG cells were maintained in DMEM+10% FBS and passaged every 3 days by TrypLE. The day before transfection, cells were inoculated to 24-well plate in 1×10⁵/cm². Plasmids of constructs V2 (P4-C2), V3 (P8-C2) and a control V1 (P0-C0) were transfected into U87 MG using Lipofectamine 3000 Transfection Reagent (Invitrogen, L3000008) following the user's guide. 72 hours after transfection, cells were collected in RIPA lysis buffer (Beyotime, P0013C) with protease inhibitor cocktail (Roche, 04693159001) and SDS-PAGE loading buffer (Cowin Bio, CW0027), denatured for 10 min at 95° C., centrifuged at 12,000 rpm for 10 min. Supernatants were separated in 4%-10% SDS-PAGE gel (Cowin Bio, CW0022M), and blotted onto 0.2 μm PVDF transfer membrane (Merck, ISEQ00010). The protein levels of GCDH and housekeeping gene GAPDH were detected by an antibody against GCDH (Abcam, ab232774) and β-TUBULIN (Proteintech, 66240-1), respectively. The Western Blot images are shown in FIG. 6.

The results as shown in FIG. 6 suggest that V1, V2 and V3 mediated similar GCDH protein expression levels in the U87 MG cells, indicating that V2 and V3 constructs could achieve comparable GCDH protein expression with the advantage of reduced CpG numbers in the coding sequences, as compared to V1.

Construct V2 was chosen for further study since V2 had a lower number of CpG than V3.

The enzymatic activities of the GCDH protein expressed by constructs V1 and V2 in the SH-sy5y cells were evaluated by incubating the cell lysates with glutaconyl-CoA and then measuring the catalytic product crotonyl-CoA by LC-MS/MS analysis,

SH-sy5y cells were maintained in DMEM+10% FBS and passaged every 3 days by TrypLE. The day before transfection, cells were inoculated to 100 mm dish in 3×10⁶/dish. Plasmids were transfected into SH-sy5y using jet Optimus reagent (Polyplus, 117-15) following the user's guide. 48 hours after transfection, cells were collected. Cells was adjusted to 3×10⁷/mL and homogenized by ultrasound in cell lysis buffer (0.2 mM Flavin adenine dinucleotide disodium salt hydrate (Sigma, F6625-25MG), 1 mM L-Cysteine (Sangon Biotech, A600132-0100) in 1×PBS (Sangon Biotech, B540626-0500)). The total protein in the cell lysis buffer was measured by BCA Protein Quantification Kit (YEASEN, 20201ES76). The GCDH activity was measured by mixing 0.5 g total protein with GCDH reaction buffer [0.15 mM Glutaryl coenzyme A lithium salt (Sigma G9510-5MG), 0.5 mM L-Cysteine, 0.1 mM Flavin adenine dinucleotide disodium salt hydrate, 1 mM Phenazine methosulfate (Sigma, P9625-1G) in 1×PBS] to a final volume of 500 μL. The reaction mixture was incubated at 37° C. for different time points: 0 min, 5 min, 10 min, 15 min respectively and terminated by 500 μL of 7M Trichloroacetic acid (Sigma, T9159-100G). The GCDH activity was measured by the increase of crotonyl-CoA. The crotonyl-CoA produced in the reaction buffer was monitored and determined by LC-MS/MS. The time course of crotonyl-CoA production is shown in FIG. 7.

As shown in FIG. 7, the GCDH protein expressed by V2 surprisingly mediated a significantly more rapid catalytic reaction as compared to the protein expressed by V1, suggesting that the optimized coding sequence of C2 expresses GCDH protein of increased enzymatic activity as compared to the protein expressed by construct V1 containing the wild-type coding sequence under the control of a CMV enhancer/promoter.

### Example 4. In Vivo Proof-of-Concept Efficacy Study

Both V1 and V2 constructs were introduced into AAV9 vector to obtain rAAV9-V1 and rAAV9-V2 for evaluation of their in vivo efficacy in the Gcdh^{-/-} mouse model.

Gcdh^{-/-} mice under normal diet showed similar life expectancy to the wild-type C57BL/6 mice. However, under a 2-day high protein diet (HPD) challenge, half of 4-week-old Gcdh^{-/-} mice would die within 3 days.

Within 24 h after birth, Gcdh^{-/-} pups were injected (single-dose) intracerebroventricularly with rAAV9-V1 or rAAV9-V2 at doses of 4.38×10⁸, 4.38×10⁹, 4.38×10¹⁰ vg, respectively. Pups injected with PBS were used as the control group. At 4 weeks post dosing, HPD was administered for 2 consecutive days, and the survival rate of each group was evaluated.

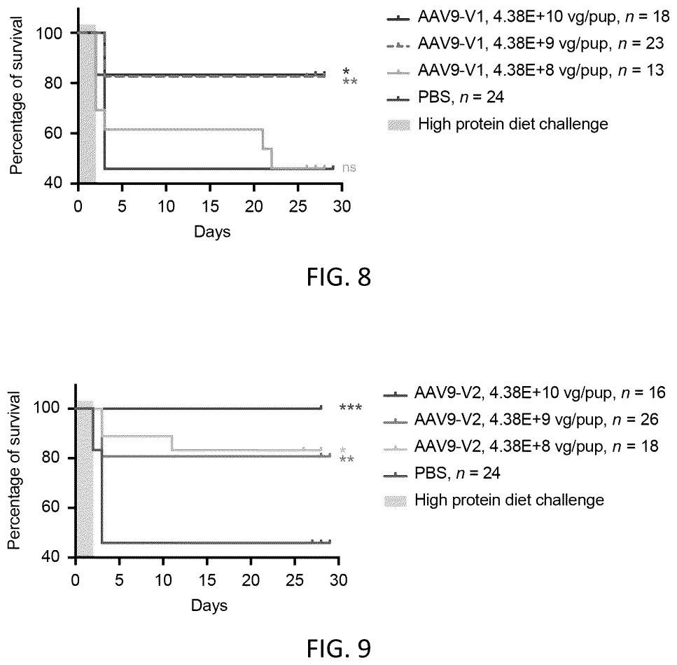

After intracerebroventricular administration of PBS, the survival rate of Gcdh^{-/-} mice under HPD challenge was 46% (FIGS. 8 and 9). After intracerebroventricular administration of rAAV9-V1 at doses of 4.38×10⁸, 4.38×10⁹, 4.38×10¹⁰ vg, respectively, the survival rates of Gcdh^{-/-} mice under HPD challenge were 46%, 83% and 83%, respectively (FIG. 8). After intracerebroventricular administration of rAAV9-V2 at doses of 4.38 10⁸, 4.38×10⁹, 4.38×10¹⁰ vg, respectively, the survival rates of Gcdh^{-/-} mice under HPD challenge were 83%, 81% and 100%, respectively (FIG. 9). Treatment with rAAV9-V2 resulted in significantly higher survival rates of the HPD-challenged Gcdh^{-/-} mice as compared to treatment with rAAV9-V1.

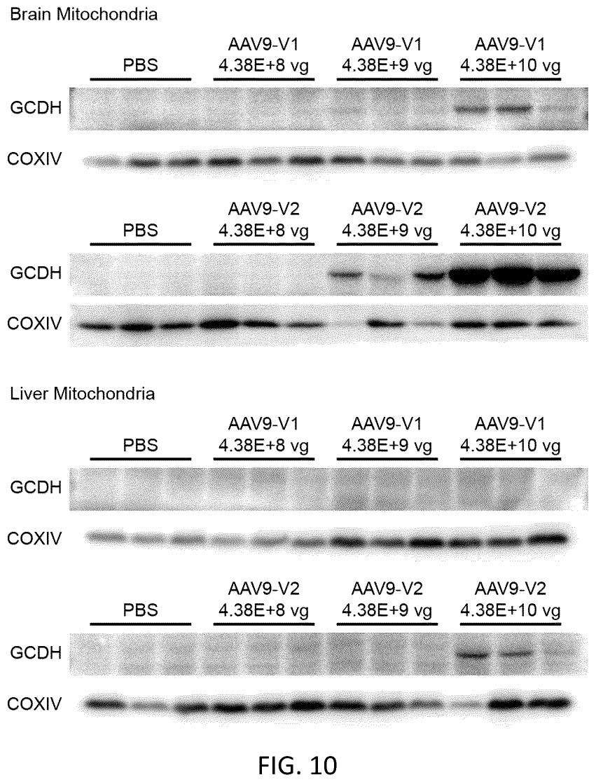

At 8 weeks after AAV administration, the surviving mice were sacrificed for brain, liver and plasma collection. Brain and liver tissues were homogenized and the mitochondria were isolated (QIAGEN, 37612). The mitochondria were collected in the RIPA lysis buffer (Beyotime, P0013C) with protease inhibitor cocktail (Roche, 04693159001) and SDS-PAGE loading buffer (Cowin Bio, CW0027), denatured for 10 min at 95° C., centrifuged at 12,000 rpm for 10 min. The supernatants were separated in 4%-10% SDS-PAGE gel (Cowin Bio, CW0022M), and blotted onto 0.2 μm PVDF transfer membrane (Merck, ISEQ00010). The protein levels of GCDH and housekeeping gene COXIV were detected by an antibody against GCDH (Abcam, ab232774) and COXIV (Abcam, ab16056), respectively. The Western Blot images are shown in FIG. 10. Brain GCDH expression was detected in a dose-dependent manner (FIG. 10). Since the mouse blood-brain barrier (BBB) was not mature at the time of AAV administration, liver GCDH expression can be detected in the highest dose group of rAAV9-V2 treatment, suggesting the AAV transferred across BBB from CNS to the peripheral tissues and organs (FIG. 10). The rAAV9-V2 mediated higher levels of GCDH protein expression than rAAV9-V1, indicating that higher levels of GCDH protein expression protected more Gcdh^{-/-} mice from HPD challenge-induced death.

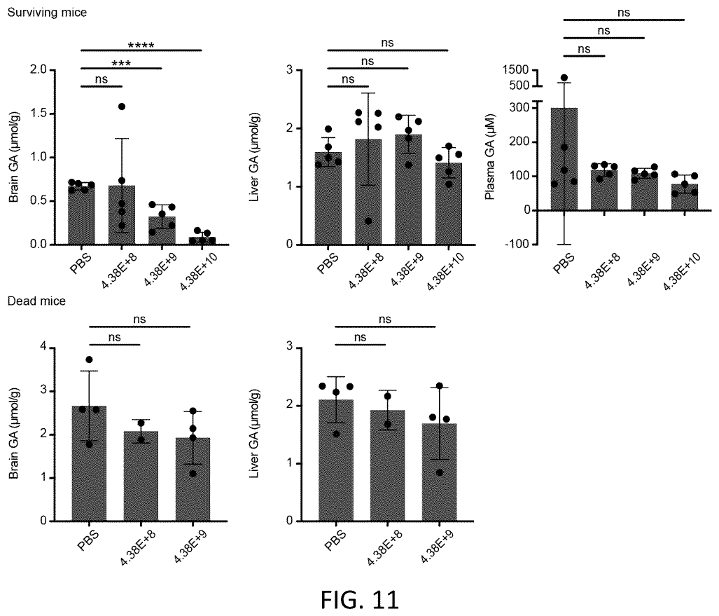

For the Gcdh^{-/-} mice that received rAAV9-V2 administration, liquid chromatography-mass spectrometry (LC-MS) analysis to measure the levels of GA showed that the GA levels significantly reduced in the brain dose-dependently (two-way ANOVA test was used for variance analysis) (FIG. 11). The liver and plasma GA levels between different treatment groups had no significant differences (FIG. 11). The brain and liver GA levels of the dead mice were also examined, and the results showed drastically elevated GA levels than the survived mice (FIG. 11). Taken the dead mice into consideration, ICV administration of rAAV9-V2 effectively reduced GA accumulation and improved the survival rate upon HPD challenge. It was also observed that the brain GA levels in the 4.38×10⁸ vg treatment group had no significant difference but the survival rate was significantly improved, indicating that even moderate GA level reduction in the brain could protect the Gcdh^{-/-} mice from acute encephalopathic crisis induced by HPD challenge, suggesting that it's more important to reduce the GA levels in the CNS than the peripheral tissues.

### Example 5. Long-Term Efficacy Study

A long-term study was conducted to test endurance of the efficacy of rAAV9-V2. AAV9-V2 was intracerebroventricularly administrated into new born Gcdh^{-/-} mice at doses of 0, 5×10⁸, 2.5×10⁹, 1×10¹⁰ vg, respectively. Unlike the study described in Example 4, no HPD challenge was used in this study. 4 weeks and 13 weeks after the AAV administration, animals were sacrificed for biochemical and histopathologic analysis.

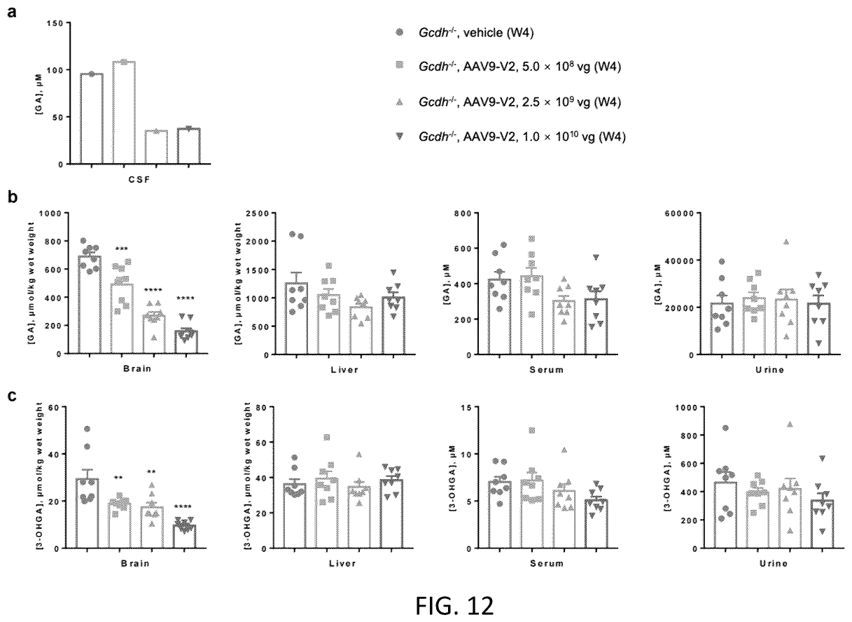

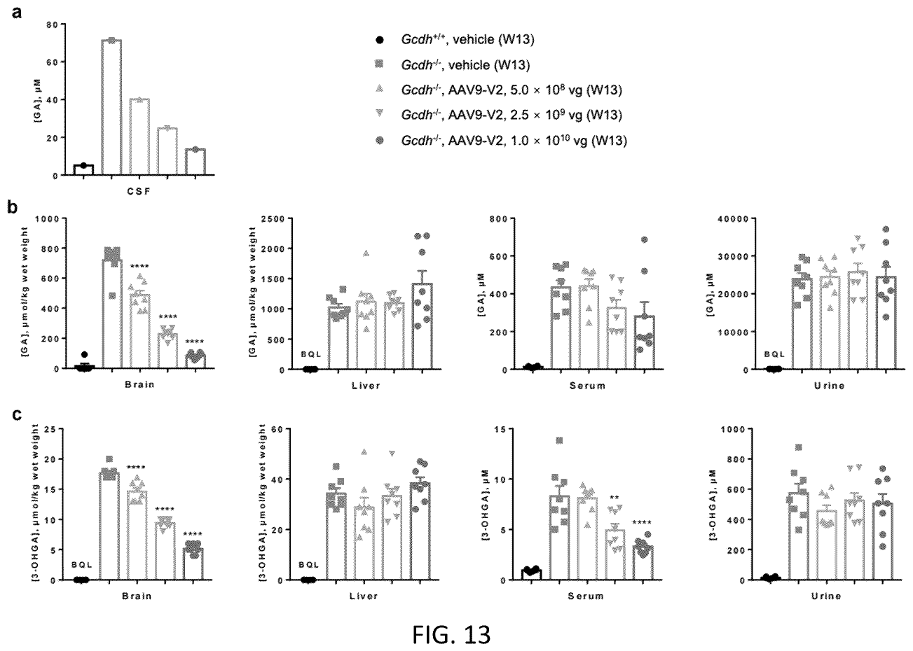

The levels of glutaric acid (GA) and 3-hydroxyglutaric acid (3-OHGA) were determined by liquid chromatography with tandem mass spectrometry (LC-MS/MS). FIG. 12 shows the results determined 4 weeks after AAV administration. As seen from FIG. 12, brain GA and 3-OHGA levels significantly decreased in a dose-dependent manner. CSF GA, serum GA and serum 3-OHGA levels showed a trend of dose-dependent decrease, yet no significance. GA and 3-OHGA levels in the liver and urine did not show obvious change. FIG. 13 shows the LC-MS/MS results measured 13 weeks after AAV administration. As seen from FIG. 13, brain GA and 3-OHGA levels significantly decreased in a dose-dependent manner. Serum GA and 3-OHGA level showed a trend of dose-dependent decrease, with the decrease of 3-OHGA level more significant. GA and 3-OHGA in the liver and urine did not show obvious change. CSF GA showed a trend of dose-dependent decrease (FIG. 13). Compared to the 4-week results, GA and 3-OHGA levels in the brain and serum, as well as GA level in the CSF showed even greater decrease at 13 weeks after AAV administration.

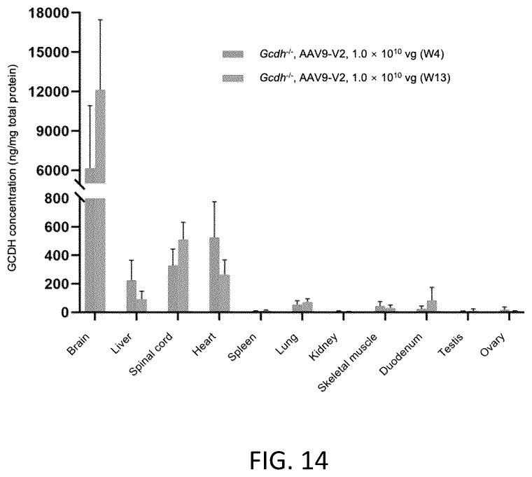

The expression of GCDH was determined by ELISA. After AAV administration, the expression of GCDH was most abundant in the brain, followed by the heart, spinal cord and liver (FIG. 14). The average brain GCDH concentration was 6,178.5 ng/mg total protein at 4 weeks after AAV administration, and 12,143.5 ng/mg total protein at 13 weeks after AAV administration. The average liver GCDH concentration was 227.0 ng/mg total protein at 4 weeks after AAV administration, and 92.2 ng/mg total protein at 13 weeks after AAV administration.

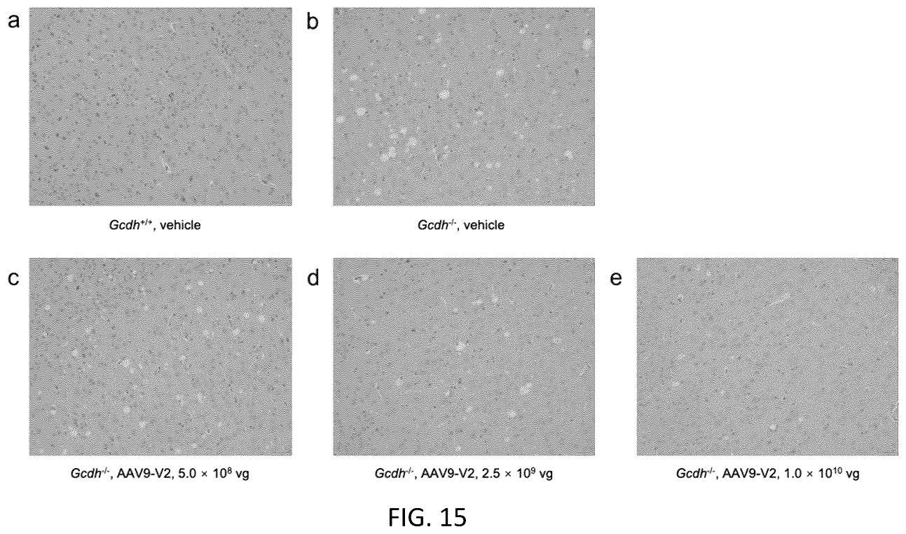

Hematoxylin-eosin (HE) staining was used for evaluation of brain histopathology. Compared to the wild type mice, vacuolation could be observed in the cortex and striatum of the Gcdh^{-/-} mice. After AAV administration, vacuolation decreased in a dose dependent manner (FIG. 15).

The results of this long term study validated the effect of AAV9-V2 in enhancing the expression of GCDH, especially in the brain, and the effects in decreasing the levels of GA and 3-OHGA in the CNS and also peripheral tissues. In addition, the results showed that long-term expression of GCDH by AAV9-V2 therapy could protect the CNS tissues from GCDH deficiency induced vacuolation. Therefore, this 13-week study provided strong evidence that AAV9-V2 could be an effective therapy for a sustained period of time.

### REFERENCES

1. Goodman S I, Moe P, Markey S P, O'brien D. Glutaric acidemia: A new disorder of amino acid metabolism. Pediatric Research. 1974; 8: 389-389.
2. Goodman S I, Markey S P, Moe P G, Miles B S, Teng C C. Glutaric aciduria; a “new” disorder of amino acid metabolism. Biochemical medicine. 1975; 12: 12-21.
3. Goodman S I, Stein D E, Schlesinger S, Christensen E, Schwartz M, Greenberg C R et al. Glutaryl-coa dehydrogenase mutations in glutaric acidemia (type i): Review and report of thirty novel mutations. Human mutation. 1998; 12: 141-144.
4. Besrat A, Polan C E, Henderson L. Mammalian metabolism of glutaric acid. Journal of Biological Chemistry. 1969; 244: 1461-1467.
5. Kolker S, Christensen E, Leonard J, Greenberg C, Burlina A, Burlina A et al. Guideline for the diagnosis and management of glutaryl-coa dehydrogenase deficiency (glutaric aciduria type i). Journal of Inherited Metabolic Disease: Official Journal of the Society for the Study of Inborn Errors of Metabolism. 2007; 30: 5-22.
6. Ullrich K, Flott-Rahmel B, SchluffP, Musshoff U, Das A, LUcke T et al. Glutaric aciduria type i: Pathomechanisms of neurodegeneration. Journal of inherited metabolic disease. 1999; 22: 392-403.
7. Sauer S W, Okun J G, Fricker G, Mahringer A, Muller I, Crnic L R et al. Intracerebral accumulation of glutaric and 3-hydroxyglutaric acids secondary to limited flux across the blood-brain barrier constitute a biochemical risk factor for neurodegeneration in glutaryl-coa dehydrogenase deficiency. J Neurochem. 2006; 97: 899-910.
8. Koeller D M, Woontner M, Crnic L S, Kleinschmidt-Demasters B, Stephens J, Hunt E L et al. Biochemical, pathologic and behavioral analysis of a mouse model of glutaric acidemia type i. Human molecular genetics. 2002; 11: 347-357.
9. Zinnanti W J, Lazovic J, Wolpert E B, Antonetti D A, Smith M B, Connor J R et al. A diet-induced mouse model for glutaric aciduria type i. Brain. 2006; 129: 899-910.
10. Bertolini T B, Shirley J L, Zolotukhin I, Li X, Kaisho T, Xiao W et al. Effect of cpg depletion of vector genome on cd8+t cell responses in aav gene therapy. Frontiers in Immunology. 2021; 12.
11. Konkle B A, Walsh C E, Escobar M A, Josephson N C, Young G, Von Drygalski A et al. Bax 335 hemophilia b gene therapy clinical trial results: Potential impact of cpg sequences on gene expression. Blood, The Journal of the American Society of Hematology. 2021; 137: 763-774.

Sequence Information
>P0
(SEQ ID NO: 1)
GACATTGATTATTGACTAGTTATTAATAGTAATCAATTACGGGGTCATTAGTTCATAGCCCATATATGGAG
TTCCGCGTTACATAACTTACGGTAAATGGCCCGCCTGGCTGACCGCCCAACGACCCCCGCCCATTGAC
GTCAATAATGACGTATGTTCCCATAGTAACGCCAATAGGGACTTTCCATTGACGTCAATGGGTGGAGTA
TTTACGGTAAACTGCCCACTTGGCAGTACATCAAGTGTATCATATGCCAAGTACGCCCCCTATTGACGT
CAATGACGGTAAATGGCCCGCCTGGCATTATGCCCAGTACATGACCTTATGGGACTTTCCTACTTGGCA
GTACATCTACGTATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAGTTTGTTTTGGC
ACCAAAATCAACGGGACTTTCCAAAATGTCGTAACAACTCCGCCCCATTGACGCAAATGGGCGGTAG
GCGTGTACGGTGGGAGGTCTATATAAGCAGAGCT
>P1
(SEQ ID NO: 2)
CGTTACATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGGCAGTACA
TCTACGTATTAGTCATCGCTATTACCATGGTCGAGGTGAGCCCCACGTTCTGCTTCACTCTCCCCATCTC
CCCCCCCTCCCCACCCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGCGGGGCGAGGCGGA
GAGGTGCGGCGGCAGCCAATCAGAGCGGCGCGCTCCGAAAGTTTCCTTTTATGGCGAGGCGGCGGCG
GCGGCGGCCCTATAAAAAGCGAAGCGCGCGGCGGGCG
>P2
(SEQ ID NO: 3)
CGTTACATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGGCAGTACA
TCTACGTATTAGTCATCGCTATTACCATGGTCGAGGTGAGCCCCACGTTCTGCTTCACTCTCCCCATCTC
CCCCCCCTCCCCACCCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGCGGGGCGAGGCGGA
GAGGTGCGGCGGCAGCCAATCAGAGCGGCGCGCTCCGAAAGTTTCCTTTTATGGCGAGGCGGCGGCG
GCGGCGGCCCTATAAAAAGCGAAGCGCGCGGCGGGCGGTAAGTTTAGTCTTTTTGTCTTTTATTTCAG
GTCCCGGATCCGGTGGTGGTGCAAATCAAAGAACTGCTCCTCAGTGGATGTTGCCTTTACTTCTAG
>P3
(SEQ ID NO: 4)
CGTTACATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGGCAGTACA
TCTACGTATTAGTCATCGCTATTACCATGGTCGAGGTGAGCCCCACGTTCTGCTTCACTCTCCCCATCTC
CCCCCCCTCCCCACCCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGCGGGGCGAGGCGGA
GAGGTGCGGCGGCAGCCAATCAGAGCGGCGCGCTCCGAAAGTTTCCTTTTATGGCGAGGCGGCGGCG
GCGGCGGCCCTATAAAAAGCGAAGCGCGCGGCGGGCGGTCAGTGTGGGGTCGGGAGTGTGGAGGGA
AGGAGGGAGGAACTGGGGGTTTAGGGACTTTCCGGGGTGACTTTCCCGTTCTGTGCTTGCAG
>P4
(SEQ ID NO: 5)
GGCTCCGGTGCCCGTCAGTGGGCAGAGCGCACATCGCCCACAGTCCCCGAGAAGTTGGGGGGAGGG
GTCGGCAATTGAACGGGTGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTAGTCGCCGTGAACGTTCTT
TTTCGCAACGGGTTTGCCGCCAGAACACAGGTCAGTGTGGGGTCGGGAGTGTGGAGGGAAGGAGGG
AGGAACTGGGGGTTTAGGGACTTTCCGGGGTGACTTTCCCGTTCTGTGCTTGCAG
>P5
(SEQ ID NO: 6)
GGCTCCGGTGCCCGTCAGTGGGCAGAGCGCACATCGCCCACAGTCCCCGAGAAGTTGGGGGGAGGG
GTCGGCAATTGAACGGGTGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTAGTCGCCGTGAACGTTCTT
TTTCGCAACGGGTTTGCCGCCAGAACACAGGTAAGGACCTCTGGTCGCACCGTGTGTCTGCTGCCCC
TGTTCAGCTGTCTGTCTGCCGCAGGTGGACTCTGTCCCAGAATCCGAGAGCTGCCCGAGCGGGGTGG
CAGGGTCGTGGCCAGGGTCAGAGGCACTAAGGCAGTGAGTGCGCTGTGCCTGCGGGGCCGGAGAAA
AGTCACCTGATCAGTCTCGCTTGCAGCTCGCACTAGCCGGGGGGCGACATGGGTGTTGGGGGGTAGG
GCTGATGAGGGTCCGAGAAGGGAGGGCACAGTGATCTTGCGGACTGGACCGAGGCGAATTCCCCTTC
CCAG
>P6
(SEQ ID NO: 7)
GGCTCCGGTGCCCGTCAGTGGGCAGAGCGCACATCGCCCACAGTCCCCGAGAAGTTGGGGGGAGGG
GTCGGCAATTGAACGGGTGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTAGTCGCCGTGAACGTTCTT
TTTCGCAACGGGTTTGCCGCCAGAACACAGGTGGGCGGGCTGGTGGGTGCCCTGAGACTGCTCCTCC
GCCTGGAGCCATAGCCACCCCACCTCAAGGCCCCTCTGTCCTTGGGGCTGGGGCTTCCTGTGGCCTAG
GCCTGGGCCTGAATTTGGGCACTGGTCCCTTTGCAG
>P7
(SEQ ID NO: 8)
GGCTCCGGTGCCCGTCAGTGGGCAGAGCGCACATCGCCCACAGTCCCCGAGAAGTTGGGGGGAGGG
GTCGGCAATTGAACGGGTGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTAGTCGCCGTGAACGTTCTT
TTTCGCAACGGGTTTGCCGCCAGAACACAGGTCAGTGTGGGGTCGGGAGTGTGGAGGGAAGGAGGG
AGGAACTGGGGGTTTAGGGACTTTCCGGGGTGACTTTCCCGTTCTGTGCTTGCAGGTAAGGACCTCT
GGTCGCACCGTGTGTCTGCTGCCCCTGTTCAGCTGTCTGTCTGCCGCAGGTGGACTCTGTCCCAGAAT
CCGAGAGCTGCCCGAGCGGGGTGGCAGGGTCGTGGCCAGGGTCAGAGGCACTAAGGCAGTGAGTGC
GCTGTGCCTGCGGGGCCGGAGAAAAGTCACCTGATCAGTCTCGCTTGCAGCTCGCACTAGCCGGGGG
GCGACATGGGTGTTGGGGGGTAGGGCTGATGAGGGTCCGAGAAGGGAGGGCACAGTGATCTTGCGG
ACTGGACCGAGGCGAATTCCCCTTCCCAG
>P8
(SEQ ID NO: 9)
GGCTCCGGTGCCCGTCAGTGGGCAGAGCGCACATCGCCCACAGTCCCCGAGAAGTTGGGGGGAGGG
GTCGGCAATTGAACGGGTGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTAGTCGCCGTGAACGTTCTT
TTTCGCAACGGGTTTGCCGCCAGAACACAGGGAGTCGCTGCGACGCTGCCTTCGCCCCGTGCCCCGC
TCCGCCGCCGCCTCGCGCCGCCCGCCCCGGCTCTGACTGACCGCGTTACTCCCACAGGTGAGCGGGC
GGGACGGCCCTTCTCCTCCGGGCTGTAATTAGCTGAGCAAGAGGTAAGGGTTTAAGGGATGGTTGGTT
GGTGGGGTATTAATGTTTAATTACCTGGAGCACCTGCCTGAAATCACTTTTTTTCAG
>C0
(SEQ ID NO: 10)
ATGGCCCTGAGAGGCGTCTCCGTGCGGCTGCTGAGCCGCGGACCCGGCCTGCACGTCCTTCGCACGT
GGGTCTCGTCGGCGGCGCAGACCGAGAAAGGCGGGAGAACACAGAGCCAACTGGCTAAGTCCTCGC
GTCCCGAGTTTGACTGGCAGGACCCGCTGGTGCTGGAGGAGCAGCTGACCACAGATGAGATCCTCAT
CAGGGACACCTTCCGCACCTACTGCCAGGAGAGACTCATGCCTCGCATCCTGTTGGCCAATCGCAAC
GAAGTTTTTCATCGGGAGATCATTTCGGAGATGGGGGAGTTGGGTGTGCTGGGCCCCACCATCAAAG
GATATGGCTGTGCTGGGGTTTCGTCTGTGGCCTATGGGCTCCTGGCCCGAGAGCTGGAGCGGGTGGAC
AGTGGCTACAGGTCGGCGATGAGTGTCCAGTCCTCCCTCGTCATGCACCCTATCTATGCCTATGGCAGC
GAGGAACAGCGGCAGAAGTACCTGCCCCAGCTGGCCAAGGGGGAGCTCCTGGGCTGCTTCGGGCTC
ACAGAGCCCAACAGCGGAAGTGACCCCAGCAGCATGGAGACCAGAGCCCACTACAACTCATCCAAC
AAGAGCTACACCCTCAATGGGACCAAGACCTGGATCACGAACTCGCCTATGGCCGATCTGTTTGTAGT
GTGGGCTCGGTGTGAAGATGGCTGCATTCGGGGCTTCCTGCTGGAGAAGGGGATGCGGGGTCTCTCG
GCCCCCAGGATCCAGGGCAAGTTCTCGCTGCGGGCCTCAGCCACAGGCATGATCATCATGGACGGTG
TGGAGGTGCCAGAGGAGAATGTGCTCCCTGGTGCATCCAGCCTGGGGGGTCCCTTCGGCTGCCTGAA
CAACGCCCGGTACGGCATCGCGTGGGGCGTGCTTGGAGCTTCGGAGTTCTGCTTGCACACAGCCCGG
CAGTACGCCCTCGACAGGATGCAGTTTGGTGTCCCACTGGCCAGGAACCAGCTGATTCAGAAGAAGC
TGGCAGACATGCTCACTGAGATTACCCTGGGCCTTCACGCCTGCCTGCAGCTCGGCCGCTTGAAGGA
CCAGGACAAGGCTGCCCCCGAGATGGTTTCTCTGCTGAAGAGGAATAACTGTGGGAAAGCCCTGGAC
ATCGCCCGCCAGGCCCGAGACATGCTGGGGGGGAATGGGATTTCTGACGAGTATCACGTGATCCGGC
ACGCCATGAACCTGGAGGCCGTGAACACCTACGAAGGTACACATGACATTCACGCCCTGATCCTTGG
GAGAGCTATCACGGGAATCCAGGCGTTCACGGCCAGCAAGTAA
>C1
(SEQ ID NO: 11)
ATGGCCCTGAGAGGCGTGTCCGTCAGACTGCTGAGCAGAGGCCCTGGCCTGCATGTGCTCAGAACCT
GGGTCAGCAGCGCTGCTCAAACAGAAAAGGGGGGCAGAACACAAAGCCAACTGGCTAAGAGCAGC
AGACCTGAATTCGATTGGCAAGACCCCCTGGTCCTGGAAGAACAGCTGACAACAGACGAGATTCTGA
TTAGAGACACATTCAGAACATATTGCCAAGAAAGACTGATGCCTAGAATCCTGCTGGCCAACAGAAAT
GAAGTGTTTCATCGGGAAATCATTAGCGAGATGGGCGAGCTGGGCGTGCTGGGCCCCACCATTAAGG
GCTACGGCTGTGCTGGGGTGTCCTCCGTGGCCTATGGCCTCCTGGCTAGAGAACTCGAAAGAGTCGAT
AGCGGCTACAGAAGCGCTATGAGCGTGCAGAGCAGCCTGGTGATGCATCCTATCTATGCTTATGGCAG
CGAAGAGCAGAGACAAAAGTATCTGCCTCAGCTGGCTAAGGGCGAGCTGCTCGGCTGCTTCGGGCTG
ACAGAACCCAATAGCGGGTCCGATCCTAGCAGCATGGAGACAAGAGCTCATTATAATAGCAGCAACA
AGAGCTATACCCTGAACGGGACAAAAACATGGATCACAAATAGCCCTATGGCTGACCTGTTTGTGGTG
TGGGCCAGATGTGAGGATGGCTGTATCAGAGGCTTTCTGCTGGAGAAGGGCATGCGGGGGCTGTCCG
CTCCTAGAATCCAAGGCAAATTTAGCCTGAGAGCTAGCGCTACAGGCATGATTATTATGGACGGCGTC
GAGGTGCCTGAGGAAAATGTGCTGCCTGGCGCTAGCAGCCTGGGCGGGCCTTTCGGCTGCCTGAATA
ACGCTAGATATGGCATCGCCTGGGGGGTGCTGGGCGCCTCCGAGTTTTGTCTGCACACAGCTAGACAG
TATGCCCTGGACAGAATGCAATTCGGGGTGCCCCTGGCTAGAAATCAGCTGATTCAAAAGAAACTGG
CTGACATGCTGACAGAAATTACACTCGGCCTCCATGCCTGTCTGCAGCTGGGCAGACTCAAAGATCA
AGATAAGGCTGCCCCTGAAATGGTCAGCCTGCTCAAAAGAAACAATTGCGGCAAAGCTCTGGATATC
GCTAGACAAGCTAGAGATATGCTCGGCGGCAACGGGATTAGCGACGAGTATCATGTGATCAGACACGC
TATGAATCTGGAAGCCGTGAACACCTATGAAGGCACACACGACATCCACGCTCTGATCCTCGGGAGA
GCTATCACCGGCATTCAAGCCTTCACAGCTAGCAAGTAA
>C2
(SEQ ID NO: 12)
ATGGCTCTGAGAGGGGTGAGCGTCAGACTGCTGAGCAGAGGCCCTGGCCTGCATGTGCTGAGAACAT
GGGTGTCCAGCGCTGCTCAGACAGAGAAGGGGGGCAGAACACAGAGCCAACTGGCCAAGAGCAGC
AGACCTGAATTTGACTGGCAAGACCCCCTGGTCCTGGAGGAGCAGCTGACCACAGATGAGATCCTGA
TCAGAGACACCTTCAGAACCTACTGCCAAGAGAGACTGATGCCTAGAATCCTGCTGGCCAACAGAAA
TGAGGTCTTCCACAGAGAAATCATTAGCGAGATGGGGGAGCTGGGGGTGCTGGGCCCTACAATCAAG
GGCTATGGCTGTGCTGGGGTGAGCAGCGTGGCCTATGGCCTGCTGGCTAGAGAGCTGGAGAGAGTGG
ACAGCGGGTACAGAAGCGCTATGAGCGTGCAGAGCAGCCTGGTCATGCACCCCATCTATGCCTATGGC
AGCGAGGAGCAGAGACAGAAATATCTCCCTCAGCTGGCCAAGGGGGAGCTGCTGGGCTGCTTTGGC
CTCACAGAGCCCAATAGCGGCAGCGACCCTAGCAGCATGGAGACAAGAGCCCACTACAACAGCAGC
AACAAGAGCTACACCCTGAATGGCACCAAGACATGGATCACAAACAGCCCCATGGCTGATCTCTTTG
TGGTCTGGGCTAGATGTGAGGATGGCTGTATCAGAGGCTTTCTCCTGGAGAAGGGCATGAGAGGCCT
GAGCGCTCCTAGAATCCAAGGCAAATTCAGCCTCAGAGCTTCCGCCACCGGGATGATCATCATGGATG
GGGTGGAGGTCCCTGAGGAGAATGTGCTGCCTGGGGCTAGCTCCCTGGGGGGCCCCTTTGGCTGTCT
CAATAATGCTAGATATGGCATTGCCTGGGGGGTGCTGGGGGCCAGCGAGTTCTGCCTGCATACAGCTA
GACAATATGCCCTGGACAGAATGCAGTTTGGGGTGCCCCTGGCTAGAAATCAGCTGATTCAGAAGAA
GCTGGCTGACATGCTGACAGAGATCACACTGGGCCTGCATGCCTGTCTGCAGCTGGGGAGACTGAAG
GACCAAGATAAGGCTGCCCCTGAGATGGTGAGCCTGCTGAAGAGAAATAACTGTGGGAAAGCTCTGG
ACATTGCTAGACAAGCTAGAGACATGCTGGGGGGCAATGGCATCTCCGATGAGTACCATGTCATCAGA
CATGCCATGAACCTGGAGGCTGTGAACACCTATGAGGGCACACATGACATCCATGCCCTGATCCTGGG
CAGAGCCATCACCGGCATCCAAGCCTTCACAGCTAGCAAGTGA
>C3
(SEQ ID NO: 13)
ATGGCCCTGAGAGGCGTCTCCGTGAGGCTGCTGAGCAGAGGACCTGGCCTGCATGTCCTTAGAACGT
GGGTCTCGTCGGCGGCGCAGACCGAGAAAGGCGGGAGAACACAGAGCCAACTGGCTAAGTCCTCGC
GTCCTGAGTTTGACTGGCAGGACCCGCTGGTGCTGGAGGAGCAGCTGACCACAGATGAGATCCTCAT
CAGGGACACCTTCAGAACCTACTGCCAGGAGAGACTCATGCCTAGAATCCTGTTGGCCAATAGAAAT
GAAGTTTTTCATAGGGAGATCATTTCGGAGATGGGGGAGTTGGGTGTGCTGGGCCCCACCATCAAAG
GATATGGCTGTGCTGGGGTTTCGTCTGTGGCCTATGGGCTCCTGGCCCGAGAGCTGGAGAGGGTGGA
CAGTGGCTACAGGTCGGCGATGAGTGTCCAGTCCTCCCTTGTCATGCACCCTATCTATGCCTATGGCAG
TGAGGAACAGAGGCAGAAGTACCTGCCCCAGCTGGCCAAGGGGGAGCTCCTGGGCTGCTTTGGGCT
CACAGAGCCCAACTCTGGAAGTGACCCCAGCAGCATGGAGACCAGAGCCCACTACAACTCATCCAA
CAAGAGCTACACCCTCAATGGGACCAAGACCTGGATCACGAACTCGCCTATGGCCGATCTGTTTGTAG
TGTGGGCTAGGTGTGAAGATGGCTGCATTAGGGGCTTCCTGCTGGAGAAGGGGATGAGGGGTCTCTC
GGCCCCCAGGATCCAGGGCAAGTTCTCGCTGAGGGCCTCAGCCACAGGCATGATCATCATGGATGGT
GTGGAGGTGCCAGAGGAGAATGTGCTCCCTGGTGCATCCAGCCTGGGGGGTCCCTTTGGCTGCCTGA
ACAATGCCAGGTATGGCATTGCGTGGGGCGTGCTTGGAGCTTCGGAGTTCTGCTTGCACACAGCCAG
GCAGTATGCCCTTGACAGGATGCAGTTTGGTGTCCCACTGGCCAGGAACCAGCTGATTCAGAAGAAG
CTGGCAGACATGCTCACTGAGATTACCCTGGGCCTTCATGCCTGCCTGCAGCTTGGCAGATTGAAGGA
CCAGGACAAGGCTGCCCCTGAGATGGTTTCTCTGCTGAAGAGGAATAACTGTGGGAAAGCCCTGGAC
ATTGCCAGACAGGCCCGAGACATGCTGGGGGGGAATGGGATTTCTGATGAGTATCATGTGATCAGGCA
TGCCATGAACCTGGAGGCCGTGAACACCTATGAAGGTACACATGACATTCATGCCCTGATCCTTGGGA
GAGCTATCACGGGAATCCAGGCGTTCACGGCCAGCAAGTAA
>C4
(SEQ ID NO: 14)
ATGGCCCTGAGAGGCGTCTCCGTGCGGCTGCTGAGCCGCGGACCCGGCCTGCACGTCCTTCGCACGT
GGGTCTCGTCGGCGGCGCAGACCGAGAAAGGCGGGAGAACACAGAGCCAACTGGCTAAGTCCTCGA
GGCCCGAGTTTGACTGGCAGGACCCCCTGGTGCTGGAGGAGCAGCTGACAACCGATGAGATCCTGAT
CAGGGATACCTTCAGAACCTACTGTCAGGAGAGGCTGATGCCCAGGATCCTGCTGGCCAACAGAAAC
GAGGTGTTCCACAGAGAGATCATCAGCGAGATGGGCGAGCTGGGCGTGCTGGGCCCTACAATCAAGG
GCTACGGCTGCGCCGGCGTGAGCAGCGTTGCCTACGGCCTGCTGGCCAGGGAGCTGGAGAGAGTGG
ATTCCGGCTACAGAAGCGCCATGAGCGTGCAGAGCTCCCTGGTCATGCACCCTATCTACGCCTACGGC
AGCGAGGAGCAGAGACAGAAGTACCTGCCCCAGCTGGCCAAAGGCGAGCTGCTGGGCTGCTTCGGC
CTGACAGAGCCTAATTCCGGCTCCGACCCCAGCTCCATGGAGACCAGAGCCCACTACAATAGCTCCA
ATAAGAGCTACACACTGAACGGCACAAAGACCTGGATCACAAACAGCCCCATGGCCGACCTGTTTGT
GGTGTGGGCCAGGTGTGAGGATGGCTGTATCAGGGGCTTTCTGCTGGAGAAGGGCATGAGAGGCCTG
TCCGCCCCCAGGATCCAGGGCAAGTTTAGCCTGAGAGCCAGCGCCACCGGCATGATCATCATGGATG
GCGTGGAGGTGCCCGAGGAGAACGTGCTGCCTGGCGCCAGCAGCCTGGGCGGACCTTTTGGCTGCC
TGAACAATGCCAGATACGGCATCGCCTGGGGCGTGCTGGGAGCCTCTGAGTTCTGCCTGCACACCGC
CAGGCAGTACGCCCTGGATAGGATGCAGTTTGGCGTGCCCCTGGCCAGAAACCAGCTGATCCAGAAG
AAGCTGGCCGACATGCTGACCGAGATCACACTGGGCCTGCACGCCTGCCTGCAGCTGGGAAGGCTGA
AGGATCAGGACAAGGCCGCCCCCGAGATGGTGTCCCTGCTGAAGAGAAATAATTGTGGCAAGGCCCT
GGACATCGCCAGACAGGCCAGAGATATGCTGGGCGGCAATGGCATCAGCGATGAGTACCACGTGATC
AGGCACGCCATGAACCTGGAGGCCGTGAACACCTACGAGGGCACCCACGACATCCACGCCCTGATCC
TGGGCAGGGCCATCACCGGCATCCAGGCCTTTACCGCCAGCAAGTAA
>C5
(SEQ ID NO: 15)
ATGGCCCTGAGAGGCGTCTCCGTGCGGCTGCTGAGCCGCGGACCCGGCCTGCACGTCCTTCGCACGT
GGGTCTCGTCGGCGGCGCAGACCGAGAAAGGCGGGAGAACACAGAGCCAACTGGCTAAGTCCTCGA
GACCCGAGTTCGACTGGCAGGACCCTCTTGTGCTGGAAGAGCAACTGACAACAGATGAGATCCTGAT
CAGAGACACCTTCAGAACCTACTGCCAGGAGAGACTGATGCCCAGAATCCTGCTGGCCAACAGAAA
CGAGGTGTTCCACAGAGAGATCATCAGCGAGATGGGCGAGCTGGGCGTGCTGGGCCCTACAATTAAA
GGATATGGCTGCGCCGGAGTGAGCAGCGTGGCTTATGGACTTCTGGCTAGAGAGCTGGAGAGAGTGG
ACAGCGGCTATAGAAGCGCCATGAGCGTGCAGAGCAGCCTGGTGATGCATCCCATTTATGCCTACGGC
AGCGAGGAGCAAAGACAGAAGTACCTGCCCCAGCTGGCCAAGGGCGAGCTGCTGGGATGTTTTGGA
CTTACAGAACCCAACAGCGGAAGCGACCCCAGCAGCATGGAAACCAGAGCTCATTATAACAGCAGCA
ACAAGAGCTACACCCTGAACGGCACCAAGACCTGGATCACCAACAGCCCCATGGCCGACCTTTTTGT
GGTGTGGGCTAGATGCGAGGACGGCTGTATTAGAGGCTTTCTGCTGGAAAAGGGCATGAGAGGCCTG
AGCGCCCCTAGAATTCAAGGCAAATTTAGCCTGAGAGCCAGCGCCACCGGAATGATTATCATGGACGG
CGTGGAGGTGCCCGAGGAGAATGTGCTGCCTGGAGCTAGCAGCCTGGGAGGCCCTTTTGGATGTCTG
AATAATGCCAGATACGGCATCGCCTGGGGCGTGCTGGGAGCTAGCGAGTTTTGTCTGCATACAGCCAG
ACAGTACGCCCTGGACAGAATGCAGTTCGGCGTGCCCCTTGCTAGAAATCAGCTGATCCAGAAGAAG
CTGGCCGACATGCTGACCGAGATCACCCTGGGACTTCACGCCTGTCTGCAACTGGGAAGACTGAAAG
ATCAGGACAAGGCCGCCCCCGAAATGGTGTCTCTGCTTAAAAGAAACAACTGCGGCAAGGCCCTGG
ACATCGCCAGACAAGCTAGAGATATGCTGGGCGGCAATGGCATTAGCGATGAATATCACGTGATTAGA
CACGCCATGAACCTGGAGGCCGTGAACACCTATGAGGGCACACATGACATCCACGCCCTGATTCTGG
GAAGAGCCATTACCGGCATCCAGGCCTTTACCGCCAGCAAGTAA
>C6
(SEQ ID NO: 16)
ATGGCCCTGAGAGGCGTCTCCGTGCGGCTGCTGAGCCGCGGACCCGGCCTGCACGTCCTTCGCACGT
GGGTCTCGTCGGCGGCGCAGACCGAGAAAGGCGGGAGAACACAGAGCCAACTGGCTAAGTCCTCGC
GTCCCGAGTTTGACTGGCAGGACCCGCTGGTGCTGGAGGAGCAGCTGACCACAGATGAGATCCTCAT
CAGGGACACCTTCCGCACCTACTGCCAGGAGAGACTCATGCCTCGCATCCTGTTGGCCAATCGCAAC
GAAGTTTTTCATCGGGAGATCATTTCGGAGATGGGGGAGTTGGGTGTGCTGGGCCCCACCATCAAAG
GATATGGCTGTGCTGGGGTTTCGTCTGTGGCCTATGGGCTCCTGGCCCGAGAGCTGGAGCGGGTGGAC
AGTGGCTACAGGTCGGCGATGAGTGTCCAGTCCTCCCTCGTCATGCACCCTATCTATGCCTATGGCAGC
GAGGAACAGCGGCAGAAGTACCTGCCCCAGCTGGCCAAGGGGGAGCTCCTGGGCTGCTTTGGGCTC
ACAGAGCCCAACTCTGGAAGTGACCCCAGCAGCATGGAGACCAGAGCCCACTACAACTCATCCAAC
AAGAGCTACACCCTCAATGGGACCAAGACCTGGATCACGAACTCGCCTATGGCCGATCTGTTTGTAGT
GTGGGCTAGGTGTGAAGATGGCTGCATTAGGGGCTTCCTGCTGGAGAAGGGGATGAGGGGTCTCTCG
GCCCCCAGGATCCAGGGCAAGTTCTCGCTGAGGGCCTCAGCCACAGGCATGATCATCATGGATGGTGT
GGAGGTGCCAGAGGAGAATGTGCTCCCTGGTGCATCCAGCCTGGGGGGTCCCTTTGGCTGCCTGAAC
AATGCCAGGTATGGCATTGCGTGGGGCGTGCTTGGAGCTTCGGAGTTCTGCTTGCACACAGCCAGGC
AGTATGCCCTTGACAGGATGCAGTTTGGTGTCCCACTGGCCAGGAACCAGCTGATTCAGAAGAAGCT
GGCAGACATGCTCACTGAGATTACCCTGGGCCTTCATGCCTGCCTGCAGCTTGGCAGATTGAAGGACC
AGGACAAGGCTGCCCCTGAGATGGTTTCTCTGCTGAAGAGGAATAACTGTGGGAAAGCCCTGGACAT
TGCCAGACAGGCCCGAGACATGCTGGGGGGGAATGGGATTTCTGATGAGTATCATGTGATCAGGCATG
CCATGAACCTGGAGGCCGTGAACACCTATGAAGGTACACATGACATTCATGCCCTGATCCTTGGGAGA
GCTATCACGGGAATCCAGGCGTTCACGGCCAGCAAGTAA
>C7
(SEQ ID NO: 17)
ATGGCCCTGAGAGGCGTCTCCGTGCGGCTGCTGAGCCGCGGACCCGGCCTGCACGTCCTTCGCACGT
GGGTCTCGTCGGCGGCGCAGACCGAGAAAGGCGGGAGAACACAGAGCCAACTGGCTAAGTCCTCGC
GTCCCGAGTTTGACTGGCAGGACCCGCTGGTGCTGGAGGAGCAGCTGACCACAGATGAGATCCTCAT
CAGGGACACCTTCCGCACCTACTGCCAGGAGAGACTCATGCCTCGCATCCTGTTGGCCAATCGCAAC
GAAGTTTTTCATCGGGAGATCATTTCGGAGATGGGGGAGTTGGGTGTGCTGGGCCCCACCATCAAAG
GATATGGCTGTGCTGGGGTTTCGTCTGTGGCCTATGGGCTCCTGGCCCGAGAGCTGGAGCGGGTGGAC
AGTGGCTACAGGTCGGCGATGAGTGTCCAGTCCTCCCTCGTCATGCACCCTATCTATGCCTATGGCAGC
GAGGAACAGCGGCAGAAGTACCTGCCCCAGCTGGCCAAGGGGGAGCTCCTGGGCTGCTTTGGCCTC
ACTGAGCCAAATTCTGGTTCAGACCCATCATCTATGGAAACAAGGGCCCATTACAATTCATCTAATAAG
TCATACACTCTGAATGGTACTAAGACCTGGATCACCAACTCTCCAATGGCAGACCTGTTTGTAGTTTGG
GCAAGATGTGAAGATGGCTGTATTAGGGGTTTCCTCCTGGAGAAGGGCATGAGAGGTCTCTCTGCACC
AAGGATTCAGGGAAAATTCTCTCTGAGAGCTTCTGCTACAGGCATGATTATTATGGATGGGGTGGAGG
TTCCTGAAGAGAATGTCCTGCCTGGAGCTTCATCACTGGGGGGCCCCTTTGGCTGTCTGAACAATGCC
AGATATGGTATTGCATGGGGGGTTCTGGGGGCTAGTGAGTTCTGCCTGCACACAGCTAGACAGTATGC
TCTGGATAGGATGCAGTTTGGTGTTCCTCTGGCTAGGAACCAGCTGATTCAGAAAAAACTGGCTGATA
TGCTCACAGAGATTACACTGGGTCTGCATGCTTGTCTCCAGCTGGGTAGACTCAAAGATCAGGATAAG
GCTGCTCCAGAAATGGTGTCACTCCTGAAGAGGAATAACTGTGGCAAGGCTCTGGACATTGCTAGAC
AGGCTAGGGATATGCTGGGTGGTAATGGCATCTCAGATGAATATCATGTTATTAGACATGCCATGAATCT
GGAGGCTGTTAACACTTATGAAGGCACACATGATATTCATGCCCTCATCCTGGGGAGAGCTATTACAG
GTATTCAGGCCTTTACTGCTTCTAAGTGA
>C8
(SEQ ID NO: 18)
ATGGCCCTGAGAGGCGTCTCCGTGCGGCTGCTGAGCCGCGGACCCGGCCTGCACGTCCTTCGCACGT
GGGTCTCGTCGGCGGCGCAGACCGAGAAAGGCGGGAGAACACAGAGCCAACTGGCTAAGTCCTCGC
GTCCCGAGTTTGACTGGCAGGACCCGCTGGTGCTGGAGGAGCAGCTGACCACAGATGAGATCCTCAT
CAGGGACACCTTCCGCACCTACTGCCAGGAGAGACTCATGCCTCGCATCCTGTTGGCCAATCGCAAC
GAAGTTTTTCATCGGGAGATCATTTCGGAGATGGGGGAGTTGGGTGTGCTGGGCCCCACCATCAAAG
GATATGGCTGTGCTGGGGTTTCGTCTGTGGCCTATGGGCTCCTGGCCCGAGAGCTGGAGCGGGTGGAC
AGTGGCTACAGGTCGGCGATGAGTGTCCAGTCCTCCCTCGTCATGCACCCTATCTATGCCTATGGCAGC
GAGGAACAGCGGCAGAAGTACCTGCCCCAGCTGGCCAAGGGGGAGCTCCTGGGCTGCTTCGGGCTC
ACAGAGCCCAACAGCGGAAGTGACCCCAGCAGCATGGAGACCAGAGCCCACTACAACTCATCCAAC
AAGAGCTACACCCTCAATGGGACCAAGACCTGGATCACGAACTCGCCTATGGCCGATCTGTTTGTAGT
GTGGGCTCGGTGTGAAGATGGCTGCATTCGGGGCTTCCTGCTGGAGAAGGGGATGCGGGGTCTCTCG
GCCCCCAGGATCCAGGGCAAGTTCTCGCTGCGGGCCTCAGCCACAGGCATGATCATCATGGACGGTG
TGGAGGTGCCAGAGGAGAATGTGCTCCCTGGTGCATCCAGCCTGGGGGGTCCCTTCGGCTGTCTGAA
CAATGCCAGATATGGTATTGCATGGGGGGTTCTGGGGGCTAGTGAGTTCTGCCTGCACACAGCTAGAC
AGTATGCTCTGGATAGGATGCAGTTTGGTGTTCCTCTGGCTAGGAACCAGCTGATTCAGAAAAAACTG
GCTGATATGCTCACAGAGATTACACTGGGTCTGCATGCTTGTCTCCAGCTGGGTAGACTCAAAGATCA
GGATAAGGCTGCTCCAGAAATGGTGTCACTCCTGAAGAGGAATAACTGTGGCAAGGCTCTGGACATT
GCTAGACAGGCTAGGGATATGCTGGGTGGTAATGGCATCTCAGATGAATATCATGTTATTAGACATGCC
ATGAATCTGGAGGCTGTTAACACTTATGAAGGCACACATGATATTCATGCCCTCATCCTGGGGAGAGCT
ATTACAGGTATTCAGGCCTTTACTGCTTCTAAGTGA
>V1
(SEQ ID NO: 19)
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGGGTTAAACGTTGACATTGATTATTGCGG
CCTCTAGACTCGAGGCGTTGACATTGATTATTGACTAGTTATTAATAGTAATCAATTACGGGGTCATTAG
TTCATAGCCCATATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAGGGACTTTCCATT
GACGTCAATGGGTGGAGTATTTACGGTAAACTGCCCACTTGGCAGTACATCAAGTGTATCATATGCCAA
GTACGCCCCCTATTGACGTCAATGACGGTAAATGGCCCGCCTGGCATTATGCCCAGTACATGACCTTAT
GGGACTTTCCTACTTGGCAGTACATCTACGTATTAGTCATCGCTATTACCATGGTGATGCGGTTTTGGCA
GTACATCAATGGGCGTGGATAGCGGTTTGACTCACGGGGATTTCCAAGTCTCCACCCCATTGACGTCA
ATGGGAGTTTGTTTTGGCACCAAAATCAACGGGACTTTCCAAAATGTCGTAACAACTCCGCCCCATTG
ACGCAAATGGGCGGTAGGCGTGTACGGTGGGAGGTCTATATAAGCAGAGCTAAGCTTGCCGCCACCAT
GGCCCTGAGAGGCGTCTCCGTGCGGCTGCTGAGCCGCGGACCCGGCCTGCACGTCCTTCGCACGTGG
GTCTCGTCGGCGGCGCAGACCGAGAAAGGCGGGAGAACACAGAGCCAACTGGCTAAGTCCTCGCGT
CCCGAGTTTGACTGGCAGGACCCGCTGGTGCTGGAGGAGCAGCTGACCACAGATGAGATCCTCATCA
GGGACACCTTCCGCACCTACTGCCAGGAGAGACTCATGCCTCGCATCCTGTTGGCCAATCGCAACGA
AGTTTTTCATCGGGAGATCATTTCGGAGATGGGGGAGTTGGGTGTGCTGGGCCCCACCATCAAAGGAT
ATGGCTGTGCTGGGGTTTCGTCTGTGGCCTATGGGCTCCTGGCCCGAGAGCTGGAGCGGGTGGACAG
TGGCTACAGGTCGGCGATGAGTGTCCAGTCCTCCCTCGTCATGCACCCTATCTATGCCTATGGCAGCGA
GGAACAGCGGCAGAAGTACCTGCCCCAGCTGGCCAAGGGGGAGCTCCTGGGCTGCTTCGGGCTCAC
AGAGCCCAACAGCGGAAGTGACCCCAGCAGCATGGAGACCAGAGCCCACTACAACTCATCCAACAA
GAGCTACACCCTCAATGGGACCAAGACCTGGATCACGAACTCGCCTATGGCCGATCTGTTTGTAGTGT
GGGCTCGGTGTGAAGATGGCTGCATTCGGGGCTTCCTGCTGGAGAAGGGGATGCGGGGTCTCTCGGC
CCCCAGGATCCAGGGCAAGTTCTCGCTGCGGGCCTCAGCCACAGGCATGATCATCATGGACGGTGTG
GAGGTGCCAGAGGAGAATGTGCTCCCTGGTGCATCCAGCCTGGGGGGTCCCTTCGGCTGCCTGAACA
ACGCCCGGTACGGCATCGCGTGGGGCGTGCTTGGAGCTTCGGAGTTCTGCTTGCACACAGCCCGGCA
GTACGCCCTCGACAGGATGCAGTTTGGTGTCCCACTGGCCAGGAACCAGCTGATTCAGAAGAAGCTG
GCAGACATGCTCACTGAGATTACCCTGGGCCTTCACGCCTGCCTGCAGCTCGGCCGCTTGAAGGACC
AGGACAAGGCTGCCCCCGAGATGGTTTCTCTGCTGAAGAGGAATAACTGTGGGAAAGCCCTGGACAT
CGCCCGCCAGGCCCGAGACATGCTGGGGGGGAATGGGATTTCTGACGAGTATCACGTGATCCGGCAC
GCCATGAACCTGGAGGCCGTGAACACCTACGAAGGTACACATGACATTCACGCCCTGATCCTTGGGA
GAGCTATCACGGGAATCCAGGCGTTCACGGCCAGCAAGTAAGAATTCCAGACATGATAAGATACATTG
ATGAGTTTGGACAAACCACAACTAGAATGCAGTGAAAAAAATGCTTTATTTGTGAAATTTGTGATGCT
ATTGCTTTATTTGTAACCATTATAAGCTGCAATAAACAAGTTAACAACAACAATTGCATTCATTTTATGT
TTCAGGTTCAGGGGGAGGTGTGGGAGGTTTTTTCGTTACTAGAGCATGGCTACGTAGATAAGTAGCAT
GGCGGGTTAATCATTAACTACAAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCGGGCGGCCTCAGT
GAGCGAGCGAGCGCGCAG
>V2
(SEQ ID NO: 20)
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGGGTTATCTAGACCTAGGACTAGTGGCTC
CGGTGCCCGTCAGTGGGCAGAGCGCACATCGCCCACAGTCCCCGAGAAGTTGGGGGGAGGGGTCGG
CAATTGAACGGGTGCCTAGAGAAGGTGGCGCGGGGTAAACTGGGAAAGTGATGTCGTGTACTGGCTC
CGCCTTTTTCCCGAGGGTGGGGGAGAACCGTATATAAGTGCAGTAGTCGCCGTGAACGTTCTTTTTCG
CAACGGGTTTGCCGCCAGAACACAGGTCAGTGTGGGGTCGGGAGTGTGGAGGGAAGGAGGGAGGA
ACTGGGGGTTTAGGGACTTTCCGGGGTGACTTTCCCGTTCTGTGCTTGCAGAAGCTTGCCGCCACCAT
GGCTCTGAGAGGGGTGAGCGTCAGACTGCTGAGCAGAGGCCCTGGCCTGCATGTGCTGAGAACATG
GGTGTCCAGCGCTGCTCAGACAGAGAAGGGGGGCAGAACACAGAGCCAACTGGCCAAGAGCAGCA
GACCTGAATTTGACTGGCAAGACCCCCTGGTCCTGGAGGAGCAGCTGACCACAGATGAGATCCTGAT
CAGAGACACCTTCAGAACCTACTGCCAAGAGAGACTGATGCCTAGAATCCTGCTGGCCAACAGAAAT
GAGGTCTTCCACAGAGAAATCATTAGCGAGATGGGGGAGCTGGGGGTGCTGGGCCCTACAATCAAGG
GCTATGGCTGTGCTGGGGTGAGCAGCGTGGCCTATGGCCTGCTGGCTAGAGAGCTGGAGAGAGTGGA
CAGCGGGTACAGAAGCGCTATGAGCGTGCAGAGCAGCCTGGTCATGCACCCCATCTATGCCTATGGCA
GCGAGGAGCAGAGACAGAAATATCTCCCTCAGCTGGCCAAGGGGGAGCTGCTGGGCTGCTTTGGCCT
CACAGAGCCCAATAGCGGCAGCGACCCTAGCAGCATGGAGACAAGAGCCCACTACAACAGCAGCAA
CAAGAGCTACACCCTGAATGGCACCAAGACATGGATCACAAACAGCCCCATGGCTGATCTCTTTGTG
GTCTGGGCTAGATGTGAGGATGGCTGTATCAGAGGCTTTCTCCTGGAGAAGGGCATGAGAGGCCTGA
GCGCTCCTAGAATCCAAGGCAAATTCAGCCTCAGAGCTTCCGCCACCGGGATGATCATCATGGATGGG
GTGGAGGTCCCTGAGGAGAATGTGCTGCCTGGGGCTAGCTCCCTGGGGGGCCCCTTTGGCTGTCTCA
ATAATGCTAGATATGGCATTGCCTGGGGGGTGCTGGGGGCCAGCGAGTTCTGCCTGCATACAGCTAGA
CAATATGCCCTGGACAGAATGCAGTTTGGGGTGCCCCTGGCTAGAAATCAGCTGATTCAGAAGAAGCT
GGCTGACATGCTGACAGAGATCACACTGGGCCTGCATGCCTGTCTGCAGCTGGGGAGACTGAAGGAC
CAAGATAAGGCTGCCCCTGAGATGGTGAGCCTGCTGAAGAGAAATAACTGTGGGAAAGCTCTGGACA
TTGCTAGACAAGCTAGAGACATGCTGGGGGGCAATGGCATCTCCGATGAGTACCATGTCATCAGACAT
GCCATGAACCTGGAGGCTGTGAACACCTATGAGGGCACACATGACATCCATGCCCTGATCCTGGGCA
GAGCCATCACCGGCATCCAAGCCTTCACAGCTAGCAAGTGAGAATTCCAGACATGATAAGATACATTG
ATGAGTTTGGACAAACCACAACTAGAATGCAGTGAAAAAAATGCTTTATTTGTGAAATTTGTGATGCT
ATTGCTTTATTTGTAACCATTATAAGCTGCAATAAACAAGTTAACAACAACAATTGCATTCAGGTTAATC
ATTAACTACAAGGAACCCCTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCTCAGTGAGCGAGCGAGC
GCGCAG
>V3
(SEQ ID NO: 21)
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGGGTTATGAATGCAATTGTTGTTGTTAAC
TTGTTTATTGCAGCTTATAATGGTTACAAATAAAGCAATAGCATCACAAATTTCACAAATAAAGCATTTT
TTTCACTGCATTCTAGTTGTGGTTTGTCCAAACTCATCAATGTATCTTATCATGTCTGGAATTCTCACTT
GCTAGCTGTGAAGGCTTGGATGCCGGTGATGGCTCTGCCCAGGATCAGGGCATGGATGTCATGTGTGC
CCTCATAGGTGTTCACAGCCTCCAGGTTCATGGCATGTCTGATGACATGGTACTCATCGGAGATGCCAT
TGCCCCCCAGCATGTCTCTAGCTTGTCTAGCAATGTCCAGAGCTTTCCCACAGTTATTTCTCTTCAGCA
GGCTCACCATCTCAGGGGCAGCCTTATCTTGGTCCTTCAGTCTCCCCAGCTGCAGACAGGCATGCAGG
CCCAGTGTGATCTCTGTCAGCATGTCAGCCAGCTTCTTCTGAATCAGCTGATTTCTAGCCAGGGGCAC
CCCAAACTGCATTCTGTCCAGGGCATATTGTCTAGCTGTATGCAGGCAGAACTCGCTGGCCCCCAGCA
CCCCCCAGGCAATGCCATATCTAGCATTATTGAGACAGCCAAAGGGGCCCCCCAGGGAGCTAGCCCCA
GGCAGCACATTCTCCTCAGGGACCTCCACCCCATCCATGATGATCATCCCGGTGGCGGAAGCTCTGAG
GCTGAATTTGCCTTGGATTCTAGGAGCGCTCAGGCCTCTCATGCCCTTCTCCAGGAGAAAGCCTCTGA
TACAGCCATCCTCACATCTAGCCCAGACCACAAAGAGATCAGCCATGGGGCTGTTTGTGATCCATGTC
TTGGTGCCATTCAGGGTGTAGCTCTTGTTGCTGCTGTTGTAGTGGGCTCTTGTCTCCATGCTGCTAGGG
TCGCTGCCGCTATTGGGCTCTGTGAGGCCAAAGCAGCCCAGCAGCTCCCCCTTGGCCAGCTGAGGGA
GATATTTCTGTCTCTGCTCCTCGCTGCCATAGGCATAGATGGGGTGCATGACCAGGCTGCTCTGCACGC
TCATAGCGCTTCTGTACCCGCTGTCCACTCTCTCCAGCTCTCTAGCCAGCAGGCCATAGGCCACGCTG
CTCACCCCAGCACAGCCATAGCCCTTGATTGTAGGGCCCAGCACCCCCAGCTCCCCCATCTCGCTAAT
GATTTCTCTGTGGAAGACCTCATTTCTGTTGGCCAGCAGGATTCTAGGCATCAGTCTCTCTTGGCAGTA
GGTTCTGAAGGTGTCTCTGATCAGGATCTCATCTGTGGTCAGCTGCTCCTCCAGGACCAGGGGGTCTT
GCCAGTCAAATTCAGGTCTGCTGCTCTTGGCCAGTTGGCTCTGTGTTCTGCCCCCCTTCTCTGTCTGA
GCAGCGCTGGACACCCATGTTCTCAGCACATGCAGGCCAGGGCCTCTGCTCAGCAGTCTGACGCTCA
CCCCTCTCAGAGCCATGGTGGCGGCAAGCTTCTGAAAAAAAGTGATTTCAGGCAGGTGCTCCAGGTA
ATTAAACATTAATACCCCACCAACCAACCATCCCTTAAACCCTTACCTCTTGCTCAGCTAATTACAGCC
CGGAGGAGAAGGGCCGTCCCGCCCGCTCACCTGTGGGAGTAACGCGGTCAGTCAGAGCCGGGGCGG
GCGGCGCGAGGCGGCGGCGGAGCGGGGCACGGGGCGAAGGCAGCGTCGCAGCGACTCCCTGTGTTC
TGGCGGCAAACCCGTTGCGAAAAAGAACGTTCACGGCGACTACTGCACTTATATACGGTTCTCCCCCA
CCCTCGGGAAAAAGGCGGAGCCAGTACACGACATCACTTTCCCAGTTTACCCCGCGCCACCTTCTCTA
GGCACCCGTTCAATTGCCGACCCCTCCCCCCAACTTCTCGGGGACTGTGGGCGATGTGCGCTCTGCCC
ACTGACGGGCACCGGAGCCACTAGTCCTAGGTCTAGAGGTTAATCATTAACTACAAGGAACCCCTAGT
GATGGAGTTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAG
>L-ITR
(SEQ ID NO: 22)
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGG
>R-ITR
(SEQ ID NO: 23)
AGGAACCCCTAGTGATGGAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAGCGAGCGCGCAG
>CMV ENHANCER
(SEQ ID NO: 24)
GACATTGATTATTGACTAGTTATTAATAGTAATCAATTACGGGGTCATTAGTTCATAGCCCATATATGGAG
TTCCGCGTTACATAACTTACGGTAAATGGCCCGCCTGGCTGACCGCCCAACGACCCCCGCCCATTGAC
GTCAATAATGACGTATGTTCCCATAGTAACGCCAATAGGGACTTTCCATTGACGTCAATGGGTGGAGTA
TTTACGGTAAACTGCCCACTTGGCAGTACATCAAGTGTATCATATGCCAAGTACGCCCCCTATTGACGT
CAATGACGGTAAATGGCCCGCCTGGCATTATGCCCAGTACATGACCTTATGGGACTTTCCTACTTGGCA
GTACATCTACGTATTAGTCATCGCTATTACCATG
>CMV PROMOTER
(SEQ ID NO: 25)
GTGATGCGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAATGTCGTA
ACAACTCCGCCCCATTGACGCAAATGGGCGGTAGGCGTGTACGGTGGGAGGTCTATATAAGCAGAGC
T
>CHICKEN B-ACTIN PROMOTER
(SEQ ID NO: 26)
TCGAGGTGAGCCCCACGTTCTGCTTCACTCTCCCCATCTCCCCCCCCTCCCCACCCCCAATTTTGTATT
TATTTATTTTTTAATTATTTTGTGCAGCGATGGGGGCGGGGGGGGGGGGGGGGCGCGCGCCAGGCGGG
GCGGGGCGGGGCGAGGGGCGGGGGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAAAGCGAAGCGCG
CGGCGGGCG
>HGCDH INTRON 1
(SEQ ID NO: 27)
GTCAGTGTGGGGTCGGGAGTGTGGAGGGAAGGAGGGAGGAACTGGGGGTTTAGGGACTTTCCGGGG
TGACTTTCCCGTTCTGTGCTTGCAG
>SV40 INTRON
(SEQ ID NO: 28)
GTAAGTTTAGTCTTTTTGTCTTTTATTTCAGGTCCCGGATCCGGTGGTGGTGCAAATCAAAGAACTGCT
CCTCAGTGGATGTTGCCTTTACTTCTAG
>EF-1A CORE PROMOTER
(SEQ ID NO: 29)
GGCTCCGGTGCCCGTCAGTGGGCAGAGCGCACATCGCCCACAGTCCCCGAGAAGTTGGGGGGAGGG
GTCGGCAATTGAACCGGTGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTAGTCGCCGTGAACGTTCTT
TTTCGCAACGGGTTTGCCGCCAGAACACAG
>HGCDH INTRON 2
(SEQ ID NO: 30)
GTAAGGACCTCTGGTCGCACCGTGTGTCTGCTGCCCCTGTTCAGCTGTCTGTCTGCCGCAGGTGGACT
CTGTCCCAGAATCCGAGAGCTGCCCGAGCGGGGTGGCAGGGTCGTGGCCAGGGTCAGAGGCACTAA
GGCAGTGAGTGCGCTGTGCCTGCGGGGCCGGAGAAAAGTCACCTGATCAGTCTCGCTTGCAGCTCGC
ACTAGCCGGGGGGCGACATGGGTGTTGGGGGGTAGGGCTGATGAGGGTCCGAGAAGGGAGGGCACA
GTGATCTTGCGGACTGGACCGAGGCGAATTCCCCTTCCCAG
>HGCDH INTRON 3
(SEQ ID NO: 31)
GTGGGCGGGCTGGTGGGTGCCCTGAGACTGCTCCTCCGCCTGGAGCCATAGCCACCCCACCTCAAGG
CCCCTCTGTCCTTGGGGCTGGGGCTTCCTGTGGCCTAGGCCTGGGCCTGAATTTGGGCACTGGTCCCT
TTGCAG
>HYBRID INTRON
(SEQ ID NO: 32)
GGAGTCGCTGCGACGCTGCCTTCGCCCCGTGCCCCGCTCCGCCGCCGCCTCGCGCCGCCCGCCCCGG
CTCTGACTGACCGCGTTACTCCCACAGGTGAGCGGGCGGGACGGCCCTTCTCCTCCGGGCTGTAATTA
GCTGAGCAAGAGGTAAGGGTTTAAGGGATGGTTGGTTGGTGGGGTATTAATGTTTAATTACCTGGAGC
ACCTGCCTGAAATCACTTTTTTTCAG
>GCDH PROT
(SEQ ID NO: 33)
MALRGVSVRLLSRGPGLHVLRTWVSSAAQTEKGGRTQSQLAKSSRPEFDWQDPLVLEEQLTTDEILIRDT
FRTYCQERLMPRILLANRNEVFHREIISEMGELGVLGPTIKGYGCAGVSSVAYGLLARELERVDSGYRSAM
SVQSSLVMHPIYAYGSEEQRQKYLPQLAKGELLGCFGLTEPNSGSDPSSMETRAHYNSSNKSYTLNGTKT
WITNSPMADLFVVWARCEDGCIRGFLLEKGMRGLSAPRIQGKFSLRASATGMIIMDGVEVPEENVLPGAS
SLGGPFGCLNNARYGIAWGVLGASEFCLHTARQYALDRMQFGVPLARNQLIQKKLADMLTEITLGLHAC
LQLGRLKDQDKAAPEMVSLLKRNNCGKALDIARQARDMLGGNGISDEYHVIRHAMNLEAVNTYEGTH
DIHALILGRAITGIQAFTASK*

1. An isolated nucleic acid molecule, comprising a nucleotide sequence selected from a group of sequences consisting of SEQ ID NOs: 11-18, wherein the nucleotide sequence encodes human glutaryl-CoA dehydrogenase (hGCDH) polypeptide having an amino acid sequence as shown in SEQ ID NO: 33.

2. An expression cassette comprising the isolated nucleic acid molecule of claim 1.

3. The expression cassette of claim 2, further comprising a promoter component which is operatively linked to the 5′ of the nucleotide sequence encoding the hGCDH polypeptide, wherein the promoter component comprises (a) a constitutive promoter, and (b) an enhancer and/or an intron-derived fragment.

4. The expression cassette of claim 3, the constitutive promoter is a CMV promoter having the nucleotide sequence as shown in SEQ ID NO: 25, a CBA promoter having the nucleotide sequence as shown in SEQ ID NO: 26, or a human elongation factor-1 alpha (EF-1α) core promoter having the nucleotide sequence as shown in SEQ ID NO: 29.

5. The expression cassette of claim 3, wherein the enhancer is a CMV enhancer having a nucleotide sequence as shown in SEQ ID NO: 24.

6. The expression cassette of claim 3, wherein the intron-derived fragment is originated from the intron of SV40, or is consisted of one or more fragments derived from one or more intronic regions of the human GCDH gene, or is a hybrid intron.

7. The expression cassette of claim 6, wherein the intron-derived fragment has a nucleotide sequence as shown in any of SEQ ID NO: 27, SEQ ID NO: 28, SEQ ID NO: 30, SEQ ID NO: 31 or SEQ ID NO: 32.

8. The expression cassette of claim 6, wherein the intron-derived fragment comprises or consists of any two or three nucleotide sequences selected from the nucleotide sequence as shown in any of SEQ ID NO: 27, 30 and 31.

9. The expression cassette of claim 3, wherein the promoter component comprises or consists of a nucleotide sequence as shown in any one of SEQ ID NOs: 2-9.

10. The expression cassette of claim 2, wherein the expression cassette comprises or consists of a nucleotide sequence as shown in SEQ ID NO: 20 or SEQ ID NO: 21.

11. A rAAV vector comprising the nucleic acid molecule of claim 1.

12. The rAAV vector of claim 11, further comprising two AAV inverted terminal repeat (ITRs), wherein the 5′ ITR has a nucleotide sequence of SEQ ID NO: 22 and the 3′ ITR has a nucleotide sequence of SEQ ID NO: 23.

13. A viral particle, comprising the rAAV vector of claim 11 packaged into an AAV capsid.

14. The viral particle of claim 13, wherein the capsid has CNS tropism.

15. The viral particle of claim 14, wherein the capsid is AAV9 or AAV PHP.B capsid.

16. A pharmaceutical composition, comprising the rAAV vectors of claim 11, and a pharmaceutically acceptable excipient.

17. A method for treating Glutaric aciduria type I (GA-I) in a subject in need thereof, comprising administering to the subject a therapeutically effective dose of rAAV vector of claim 11.

18. The method of claim 17, wherein the subject is human.

19. The method of claim 17, wherein the rAAV vector is administered to a peripheral tissue and/or to nervous system.

20. The method of claim 19, wherein the administration to a peripheral tissue is performed via intravenous (IV), intramuscular (IM), subcutaneous (SC), intra-arterial, intraperitoneal (IP), intradermal, transdermal, oral, nasal or rectal route; and/or
the administration to nervous system is performed via intracerebroventricular (ICV), intrathecal, or intracisterna magna (ICM) route.


SEQUENCE LISTING (33 sequences)

SEQ ID NO: 1 - DNA, 584 bp, synthetic construct
  misc_feature 1..584: note = P0
  source 1..584: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 2 - DNA, 583 bp, synthetic construct
  misc_feature 1..583: note = P1
  source 1..583: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 3 - DNA, 680 bp, synthetic construct
  misc_feature 1..680: note = P2
  source 1..680: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggtaagtttagtctttttgtcttttatttcaggtcccggatccggtggtggtgcaaatcaaagaactgctcctcagtggatgttgcctttacttctag

SEQ ID NO: 4 - DNA, 674 bp, synthetic construct
  misc_feature 1..674: note = P3
  source 1..674: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggtcagtgtggggtcgggagtgtggagggaaggagggaggaactgggggtttagggactttccggggtgactttcccgttctgtgcttgcag

SEQ ID NO: 5 - DNA, 322 bp, synthetic construct
  misc_feature 1..322: note = P4
  source 1..322: mol_type = other DNA
ggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagtgtggggtcgggagtgtggagggaaggagggaggaactgggggtttagggactttccggggtgactttcccgttctgtgcttgcag

SEQ ID NO: 6 - DNA, 539 bp, synthetic construct
  misc_feature 1..539: note = P5
  source 1..539: mol_type = other DNA
ggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaaggacctctggtcgcaccgtgtgtctgctgcccctgttcagctgtctgtctgccgcaggtggactctgtcccagaatccgagagctgcccgagcggggtggcagggtcgtggccagggtcagaggcactaaggcagtgagtgcgctgtgcctgcggggccggagaaaagtcacctgatcagtctcgcttgcagctcgcactagccggggggcgacatgggtgttggggggtagggctgatgagggtccgagaagggagggcacagtgatcttgcggactggaccgaggcgaattccccttcccag

SEQ ID NO: 7 - DNA, 372 bp, synthetic construct
  misc_feature 1..372: note = P6
  source 1..372: mol_type = other DNA
ggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgggcgggctggtgggtgccctgagactgctcctccgcctggagccatagccaccccacctcaaggcccctctgtccttggggctggggcttcctgtggcctaggcctgggcctgaatttgggcactggtccctttgcag

SEQ ID NO: 8 - DNA, 630 bp, synthetic construct
  misc_feature 1..630: note = P7
  source 1..630: mol_type = other DNA
ggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagtgtggggtcgggagtgtggagggaaggagggaggaactgggggtttagggactttccggggtgactttcccgttctgtgcttgcaggtaaggacctctggtcgcaccgtgtgtctgctgcccctgttcagctgtctgtctgccgcaggtggactctgtcccagaatccgagagctgcccgagcggggtggcagggtcgtggccagggtcagaggcactaaggcagtgagtgcgctgtgcctgcggggccggagaaaagtcacctgatcagtctcgcttgcagctcgcactagccggggggcgacatgggtgttggggggtagggctgatgagggtccgagaagggagggcacagtgatcttgcggactggaccgaggcgaattccccttcccag

SEQ ID NO: 9 - DNA, 460 bp, synthetic construct
  misc_feature 1..460: note = P8
  source 1..460: mol_type = other DNA
ggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 10 - DNA, 1317 bp, Homo sapiens
  source 1..1317: mol_type = genomic DNA
atggccctgagaggcgtctccgtgcggctgctgagccgcggacccggcctgcacgtccttcgcacgtgggtctcgtcggcggcgcagaccgagaaaggcgggagaacacagagccaactggctaagtcctcgcgtcccgagtttgactggcaggacccgctggtgctggaggagcagctgaccacagatgagatcctcatcagggacaccttccgcacctactgccaggagagactcatgcctcgcatcctgttggccaatcgcaacgaagtttttcatcgggagatcatttcggagatgggggagttgggtgtgctgggccccaccatcaaaggatatggctgtgctggggtttcgtctgtggcctatgggctcctggcccgagagctggagcgggtggacagtggctacaggtcggcgatgagtgtccagtcctccctcgtcatgcaccctatctatgcctatggcagcgaggaacagcggcagaagtacctgccccagctggccaagggggagctcctgggctgcttcgggctcacagagcccaacagcggaagtgaccccagcagcatggagaccagagcccactacaactcatccaacaagagctacaccctcaatgggaccaagacctggatcacgaactcgcctatggccgatctgtttgtagtgtgggctcggtgtgaagatggctgcattcggggcttcctgctggagaaggggatgcggggtctctcggcccccaggatccagggcaagttctcgctgcgggcctcagccacaggcatgatcatcatggacggtgtggaggtgccagaggagaatgtgctccctggtgcatccagcctggggggtcccttcggctgcctgaacaacgcccggtacggcatcgcgtggggcgtgcttggagcttcggagttctgcttgcacacagcccggcagtacgccctcgacaggatgcagtttggtgtcccactggccaggaaccagctgattcagaagaagctggcagacatgctcactgagattaccctgggccttcacgcctgcctgcagctcggccgcttgaaggaccaggacaaggctgcccccgagatggtttctctgctgaagaggaataactgtgggaaagccctggacatcgcccgccaggcccgagacatgctgggggggaatgggatttctgacgagtatcacgtgatccggcacgccatgaacctggaggccgtgaacacctacgaaggtacacatgacattcacgccctgatccttgggagagctatcacgggaatccaggcgttcacggccagcaagtaa

SEQ ID NO: 11 - DNA, 1317 bp, synthetic construct
  misc_feature 1..1317: note = C1
  source 1..1317: mol_type = other DNA
atggccctgagaggcgtgtccgtcagactgctgagcagaggccctggcctgcatgtgctcagaacctgggtcagcagcgctgctcaaacagaaaaggggggcagaacacaaagccaactggctaagagcagcagacctgaattcgattggcaagaccccctggtcctggaagaacagctgacaacagacgagattctgattagagacacattcagaacatattgccaagaaagactgatgcctagaatcctgctggccaacagaaatgaagtgtttcatcgggaaatcattagcgagatgggcgagctgggcgtgctgggccccaccattaagggctacggctgtgctggggtgtcctccgtggcctatggcctcctggctagagaactcgaaagagtcgatagcggctacagaagcgctatgagcgtgcagagcagcctggtgatgcatcctatctatgcttatggcagcgaagagcagagacaaaagtatctgcctcagctggctaagggcgagctgctcggctgcttcgggctgacagaacccaatagcgggtccgatcctagcagcatggagacaagagctcattataatagcagcaacaagagctataccctgaacgggacaaaaacatggatcacaaatagccctatggctgacctgtttgtggtgtgggccagatgtgaggatggctgtatcagaggctttctgctggagaagggcatgcgggggctgtccgctcctagaatccaaggcaaatttagcctgagagctagcgctacaggcatgattattatggacggcgtcgaggtgcctgaggaaaatgtgctgcctggcgctagcagcctgggcgggcctttcggctgcctgaataacgctagatatggcatcgcctggggggtgctgggcgcctccgagttttgtctgcacacagctagacagtatgccctggacagaatgcaattcggggtgcccctggctagaaatcagctgattcaaaagaaactggctgacatgctgacagaaattacactcggcctccatgcctgtctgcagctgggcagactcaaagatcaagataaggctgcccctgaaatggtcagcctgctcaaaagaaacaattgcggcaaagctctggatatcgctagacaagctagagatatgctcggcggcaacgggattagcgacgagtatcatgtgatcagacacgctatgaatctggaagccgtgaacacctatgaaggcacacacgacatccacgctctgatcctcgggagagctatcaccggcattcaagccttcacagctagcaagtaa

SEQ ID NO: 12 - DNA, 1317 bp, synthetic construct
  misc_feature 1..1317: note = C2
  source 1..1317: mol_type = other DNA
atggctctgagaggggtgagcgtcagactgctgagcagaggccctggcctgcatgtgctgagaacatgggtgtccagcgctgctcagacagagaaggggggcagaacacagagccaactggccaagagcagcagacctgaatttgactggcaagaccccctggtcctggaggagcagctgaccacagatgagatcctgatcagagacaccttcagaacctactgccaagagagactgatgcctagaatcctgctggccaacagaaatgaggtcttccacagagaaatcattagcgagatgggggagctgggggtgctgggccctacaatcaagggctatggctgtgctggggtgagcagcgtggcctatggcctgctggctagagagctggagagagtggacagcgggtacagaagcgctatgagcgtgcagagcagcctggtcatgcaccccatctatgcctatggcagcgaggagcagagacagaaatatctccctcagctggccaagggggagctgctgggctgctttggcctcacagagcccaatagcggcagcgaccctagcagcatggagacaagagcccactacaacagcagcaacaagagctacaccctgaatggcaccaagacatggatcacaaacagccccatggctgatctctttgtggtctgggctagatgtgaggatggctgtatcagaggctttctcctggagaagggcatgagaggcctgagcgctcctagaatccaaggcaaattcagcctcagagcttccgccaccgggatgatcatcatggatggggtggaggtccctgaggagaatgtgctgcctggggctagctccctggggggcccctttggctgtctcaataatgctagatatggcattgcctggggggtgctgggggccagcgagttctgcctgcatacagctagacaatatgccctggacagaatgcagtttggggtgcccctggctagaaatcagctgattcagaagaagctggctgacatgctgacagagatcacactgggcctgcatgcctgtctgcagctggggagactgaaggaccaagataaggctgcccctgagatggtgagcctgctgaagagaaataactgtgggaaagctctggacattgctagacaagctagagacatgctggggggcaatggcatctccgatgagtaccatgtcatcagacatgccatgaacctggaggctgtgaacacctatgagggcacacatgacatccatgccctgatcctgggcagagccatcaccggcatccaagccttcacagctagcaagtga

SEQ ID NO: 13 - DNA, 1317 bp, synthetic construct
  misc_feature 1..1317: note = C3
  source 1..1317: mol_type = other DNA
atggccctgagaggcgtctccgtgaggctgctgagcagaggacctggcctgcatgtccttagaacgtgggtctcgtcggcggcgcagaccgagaaaggcgggagaacacagagccaactggctaagtcctcgcgtcctgagtttgactggcaggacccgctggtgctggaggagcagctgaccacagatgagatcctcatcagggacaccttcagaacctactgccaggagagactcatgcctagaatcctgttggccaatagaaatgaagtttttcatagggagatcatttcggagatgggggagttgggtgtgctgggccccaccatcaaaggatatggctgtgctggggtttcgtctgtggcctatgggctcctggcccgagagctggagagggtggacagtggctacaggtcggcgatgagtgtccagtcctcccttgtcatgcaccctatctatgcctatggcagtgaggaacagaggcagaagtacctgccccagctggccaagggggagctcctgggctgctttgggctcacagagcccaactctggaagtgaccccagcagcatggagaccagagcccactacaactcatccaacaagagctacaccctcaatgggaccaagacctggatcacgaactcgcctatggccgatctgtttgtagtgtgggctaggtgtgaagatggctgcattaggggcttcctgctggagaaggggatgaggggtctctcggcccccaggatccagggcaagttctcgctgagggcctcagccacaggcatgatcatcatggatggtgtggaggtgccagaggagaatgtgctccctggtgcatccagcctggggggtccctttggctgcctgaacaatgccaggtatggcattgcgtggggcgtgcttggagcttcggagttctgcttgcacacagccaggcagtatgcccttgacaggatgcagtttggtgtcccactggccaggaaccagctgattcagaagaagctggcagacatgctcactgagattaccctgggccttcatgcctgcctgcagcttggcagattgaaggaccaggacaaggctgcccctgagatggtttctctgctgaagaggaataactgtgggaaagccctggacattgccagacaggcccgagacatgctgggggggaatgggatttctgatgagtatcatgtgatcaggcatgccatgaacctggaggccgtgaacacctatgaaggtacacatgacattcatgccctgatccttgggagagctatcacgggaatccaggcgttcacggccagcaagtaa

SEQ ID NO: 14 - DNA, 1317 bp, synthetic construct
  misc_feature 1..1317: note = C4
  source 1..1317: mol_type = other DNA
atggccctgagaggcgtctccgtgcggctgctgagccgcggacccggcctgcacgtccttcgcacgtgggtctcgtcggcggcgcagaccgagaaaggcgggagaacacagagccaactggctaagtcctcgaggcccgagtttgactggcaggaccccctggtgctggaggagcagctgacaaccgatgagatcctgatcagggataccttcagaacctactgtcaggagaggctgatgcccaggatcctgctggccaacagaaacgaggtgttccacagagagatcatcagcgagatgggcgagctgggcgtgctgggccctacaatcaagggctacggctgcgccggcgtgagcagcgttgcctacggcctgctggccagggagctggagagagtggattccggctacagaagcgccatgagcgtgcagagctccctggtcatgcaccctatctacgcctacggcagcgaggagcagagacagaagtacctgccccagctggccaaaggcgagctgctgggctgcttcggcctgacagagcctaattccggctccgaccccagctccatggagaccagagcccactacaatagctccaataagagctacacactgaacggcacaaagacctggatcacaaacagccccatggccgacctgtttgtggtgtgggccaggtgtgaggatggctgtatcaggggctttctgctggagaagggcatgagaggcctgtccgcccccaggatccagggcaagtttagcctgagagccagcgccaccggcatgatcatcatggatggcgtggaggtgcccgaggagaacgtgctgcctggcgccagcagcctgggcggaccttttggctgcctgaacaatgccagatacggcatcgcctggggcgtgctgggagcctctgagttctgcctgcacaccgccaggcagtacgccctggataggatgcagtttggcgtgcccctggccagaaaccagctgatccagaagaagctggccgacatgctgaccgagatcacactgggcctgcacgcctgcctgcagctgggaaggctgaaggatcaggacaaggccgcccccgagatggtgtccctgctgaagagaaataattgtggcaaggccctggacatcgccagacaggccagagatatgctgggcggcaatggcatcagcgatgagtaccacgtgatcaggcacgccatgaacctggaggccgtgaacacctacgagggcacccacgacatccacgccctgatcctgggcagggccatcaccggcatccaggcctttaccgccagcaagtaa

SEQ ID NO: 15 - DNA, 1317 bp, synthetic construct
  misc_feature 1..1317: note = C5
  source 1..1317: mol_type = other DNA
atggccctgagaggcgtctccgtgcggctgctgagccgcggacccggcctgcacgtccttcgcacgtgggtctcgtcggcggcgcagaccgagaaaggcgggagaacacagagccaactggctaagtcctcgagacccgagttcgactggcaggaccctcttgtgctggaagagcaactgacaacagatgagatcctgatcagagacaccttcagaacctactgccaggagagactgatgcccagaatcctgctggccaacagaaacgaggtgttccacagagagatcatcagcgagatgggcgagctgggcgtgctgggccctacaattaaaggatatggctgcgccggagtgagcagcgtggcttatggacttctggctagagagctggagagagtggacagcggctatagaagcgccatgagcgtgcagagcagcctggtgatgcatcccatttatgcctacggcagcgaggagcaaagacagaagtacctgccccagctggccaagggcgagctgctgggatgttttggacttacagaacccaacagcggaagcgaccccagcagcatggaaaccagagctcattataacagcagcaacaagagctacaccctgaacggcaccaagacctggatcaccaacagccccatggccgacctttttgtggtgtgggctagatgcgaggacggctgtattagaggctttctgctggaaaagggcatgagaggcctgagcgcccctagaattcaaggcaaatttagcctgagagccagcgccaccggaatgattatcatggacggcgtggaggtgcccgaggagaatgtgctgcctggagctagcagcctgggaggcccttttggatgtctgaataatgccagatacggcatcgcctggggcgtgctgggagctagcgagttttgtctgcatacagccagacagtacgccctggacagaatgcagttcggcgtgccccttgctagaaatcagctgatccagaagaagctggccgacatgctgaccgagatcaccctgggacttcacgcctgtctgcaactgggaagactgaaagatcaggacaaggccgcccccgaaatggtgtctctgcttaaaagaaacaactgcggcaaggccctggacatcgccagacaagctagagatatgctgggcggcaatggcattagcgatgaatatcacgtgattagacacgccatgaacctggaggccgtgaacacctatgagggcacacatgacatccacgccctgattctgggaagagccattaccggcatccaggcctttaccgccagcaagtaa

SEQ ID NO: 16 - DNA, 1317 bp, synthetic construct
  misc_feature 1..1317: note = C6
  source 1..1317: mol_type = other DNA
atggccctgagaggcgtctccgtgcggctgctgagccgcggacccggcctgcacgtccttcgcacgtgggtctcgtcggcggcgcagaccgagaaaggcgggagaacacagagccaactggctaagtcctcgcgtcccgagtttgactggcaggacccgctggtgctggaggagcagctgaccacagatgagatcctcatcagggacaccttccgcacctactgccaggagagactcatgcctcgcatcctgttggccaatcgcaacgaagtttttcatcgggagatcatttcggagatgggggagttgggtgtgctgggccccaccatcaaaggatatggctgtgctggggtttcgtctgtggcctatgggctcctggcccgagagctggagcgggtggacagtggctacaggtcggcgatgagtgtccagtcctccctcgtcatgcaccctatctatgcctatggcagcgaggaacagcggcagaagtacctgccccagctggccaagggggagctcctgggctgctttgggctcacagagcccaactctggaagtgaccccagcagcatggagaccagagcccactacaactcatccaacaagagctacaccctcaatgggaccaagacctggatcacgaactcgcctatggccgatctgtttgtagtgtgggctaggtgtgaagatggctgcattaggggcttcctgctggagaaggggatgaggggtctctcggcccccaggatccagggcaagttctcgctgagggcctcagccacaggcatgatcatcatggatggtgtggaggtgccagaggagaatgtgctccctggtgcatccagcctggggggtccctttggctgcctgaacaatgccaggtatggcattgcgtggggcgtgcttggagcttcggagttctgcttgcacacagccaggcagtatgcccttgacaggatgcagtttggtgtcccactggccaggaaccagctgattcagaagaagctggcagacatgctcactgagattaccctgggccttcatgcctgcctgcagcttggcagattgaaggaccaggacaaggctgcccctgagatggtttctctgctgaagaggaataactgtgggaaagccctggacattgccagacaggcccgagacatgctgggggggaatgggatttctgatgagtatcatgtgatcaggcatgccatgaacctggaggccgtgaacacctatgaaggtacacatgacattcatgccctgatccttgggagagctatcacgggaatccaggcgttcacggccagcaagtaa

SEQ ID NO: 17 - DNA, 1317 bp, synthetic construct
  misc_feature 1..1317: note = C7
  source 1..1317: mol_type = other DNA
atggccctgagaggcgtctccgtgcggctgctgagccgcggacccggcctgcacgtccttcgcacgtgggtctcgtcggcggcgcagaccgagaaaggcgggagaacacagagccaactggctaagtcctcgcgtcccgagtttgactggcaggacccgctggtgctggaggagcagctgaccacagatgagatcctcatcagggacaccttccgcacctactgccaggagagactcatgcctcgcatcctgttggccaatcgcaacgaagtttttcatcgggagatcatttcggagatgggggagttgggtgtgctgggccccaccatcaaaggatatggctgtgctggggtttcgtctgtggcctatgggctcctggcccgagagctggagcgggtggacagtggctacaggtcggcgatgagtgtccagtcctccctcgtcatgcaccctatctatgcctatggcagcgaggaacagcggcagaagtacctgccccagctggccaagggggagctcctgggctgctttggcctcactgagccaaattctggttcagacccatcatctatggaaacaagggcccattacaattcatctaataagtcatacactctgaatggtactaagacctggatcaccaactctccaatggcagacctgtttgtagtttgggcaagatgtgaagatggctgtattaggggtttcctcctggagaagggcatgagaggtctctctgcaccaaggattcagggaaaattctctctgagagcttctgctacaggcatgattattatggatggggtggaggttcctgaagagaatgtcctgcctggagcttcatcactggggggcccctttggctgtctgaacaatgccagatatggtattgcatggggggttctgggggctagtgagttctgcctgcacacagctagacagtatgctctggataggatgcagtttggtgttcctctggctaggaaccagctgattcagaaaaaactggctgatatgctcacagagattacactgggtctgcatgcttgtctccagctgggtagactcaaagatcaggataaggctgctccagaaatggtgtcactcctgaagaggaataactgtggcaaggctctggacattgctagacaggctagggatatgctgggtggtaatggcatctcagatgaatatcatgttattagacatgccatgaatctggaggctgttaacacttatgaaggcacacatgatattcatgccctcatcctggggagagctattacaggtattcaggcctttactgcttctaagtga

SEQ ID NO: 18 - DNA, 1317 bp, synthetic construct
  misc_feature 1..1317: note = C8
  source 1..1317: mol_type = other DNA
atggccctgagaggcgtctccgtgcggctgctgagccgcggacccggcctgcacgtccttcgcacgtgggtctcgtcggcggcgcagaccgagaaaggcgggagaacacagagccaactggctaagtcctcgcgtcccgagtttgactggcaggacccgctggtgctggaggagcagctgaccacagatgagatcctcatcagggacaccttccgcacctactgccaggagagactcatgcctcgcatcctgttggccaatcgcaacgaagtttttcatcgggagatcatttcggagatgggggagttgggtgtgctgggccccaccatcaaaggatatggctgtgctggggtttcgtctgtggcctatgggctcctggcccgagagctggagcgggtggacagtggctacaggtcggcgatgagtgtccagtcctccctcgtcatgcaccctatctatgcctatggcagcgaggaacagcggcagaagtacctgccccagctggccaagggggagctcctgggctgcttcgggctcacagagcccaacagcggaagtgaccccagcagcatggagaccagagcccactacaactcatccaacaagagctacaccctcaatgggaccaagacctggatcacgaactcgcctatggccgatctgtttgtagtgtgggctcggtgtgaagatggctgcattcggggcttcctgctggagaaggggatgcggggtctctcggcccccaggatccagggcaagttctcgctgcgggcctcagccacaggcatgatcatcatggacggtgtggaggtgccagaggagaatgtgctccctggtgcatccagcctggggggtcccttcggctgtctgaacaatgccagatatggtattgcatggggggttctgggggctagtgagttctgcctgcacacagctagacagtatgctctggataggatgcagtttggtgttcctctggctaggaaccagctgattcagaaaaaactggctgatatgctcacagagattacactgggtctgcatgcttgtctccagctgggtagactcaaagatcaggataaggctgctccagaaatggtgtcactcctgaagaggaataactgtggcaaggctctggacattgctagacaggctagggatatgctgggtggtaatggcatctcagatgaatatcatgttattagacatgccatgaatctggaggctgttaacacttatgaaggcacacatgatattcatgccctcatcctggggagagctattacaggtattcaggcctttactgcttctaagtga

SEQ ID NO: 19 - DNA, 2454 bp, synthetic construct
  misc_feature 1..2454: note = V1
  source 1..2454: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacgttgacattgattattgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctaagcttgccgccaccatggccctgagaggcgtctccgtgcggctgctgagccgcggacccggcctgcacgtccttcgcacgtgggtctcgtcggcggcgcagaccgagaaaggcgggagaacacagagccaactggctaagtcctcgcgtcccgagtttgactggcaggacccgctggtgctggaggagcagctgaccacagatgagatcctcatcagggacaccttccgcacctactgccaggagagactcatgcctcgcatcctgttggccaatcgcaacgaagtttttcatcgggagatcatttcggagatgggggagttgggtgtgctgggccccaccatcaaaggatatggctgtgctggggtttcgtctgtggcctatgggctcctggcccgagagctggagcgggtggacagtggctacaggtcggcgatgagtgtccagtcctccctcgtcatgcaccctatctatgcctatggcagcgaggaacagcggcagaagtacctgccccagctggccaagggggagctcctgggctgcttcgggctcacagagcccaacagcggaagtgaccccagcagcatggagaccagagcccactacaactcatccaacaagagctacaccctcaatgggaccaagacctggatcacgaactcgcctatggccgatctgtttgtagtgtgggctcggtgtgaagatggctgcattcggggcttcctgctggagaaggggatgcggggtctctcggcccccaggatccagggcaagttctcgctgcgggcctcagccacaggcatgatcatcatggacggtgtggaggtgccagaggagaatgtgctccctggtgcatccagcctggggggtcccttcggctgcctgaacaacgcccggtacggcatcgcgtggggcgtgcttggagcttcggagttctgcttgcacacagcccggcagtacgccctcgacaggatgcagtttggtgtcccactggccaggaaccagctgattcagaagaagctggcagacatgctcactgagattaccctgggccttcacgcctgcctgcagctcggccgcttgaaggaccaggacaaggctgcccccgagatggtttctctgctgaagaggaataactgtgggaaagccctggacatcgcccgccaggcccgagacatgctgggggggaatgggatttctgacgagtatcacgtgatccggcacgccatgaacctggaggccgtgaacacctacgaaggtacacatgacattcacgccctgatccttgggagagctatcacgggaatccaggcgttcacggccagcaagtaagaattccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcgttactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - DNA, 2088 bp, synthetic construct
  misc_feature 1..2088: note = V2
  source 1..2088: mol_type = other DNA
ctgcgcgctcgctcgctcactgaggccgcccgggcaaagcccgggcgtcgggcgacctttggtcgcccggcctcagtgagcgagcgagcgcgcagagagggagtggggttatctagacctaggactagtggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagtgtggggtcgggagtgtggagggaaggagggaggaactgggggtttagggactttccggggtgactttcccgttctgtgcttgcagaagcttgccgccaccatggctctgagaggggtgagcgtcagactgctgagcagaggccctggcctgcatgtgctgagaacatgggtgtccagcgctgctcagacagagaaggggggcagaacacagagccaactggccaagagcagcagacctgaatttgactggcaagaccccctggtcctggaggagcagctgaccacagatgagatcctgatcagagacaccttcagaacctactgccaagagagactgatgcctagaatcctgctggccaacagaaatgaggtcttccacagagaaatcattagcgagatgggggagctgggggtgctgggccctacaatcaagggctatggctgtgctggggtgagcagcgtggcctatggcctgctggctagagagctggagagagtggacagcgggtacagaagcgctatgagcgtgcagagcagcctggtcatgcaccccatctatgcctatggcagcgaggagcagagacagaaatatctccctcagctggccaagggggagctgctgggctgctttggcctcacagagcccaatagcggcagcgaccctagcagcatggagacaagagcccactacaacagcagcaacaagagctacaccctgaatggcaccaagacatggatcacaaacagccccatggctgatctctttgtggtctgggctagatgtgaggatggctgtatcagaggctttctcctggagaagggcatgagaggcctgagcgctcctagaatccaaggcaaattcagcctcagagcttccgccaccgggatgatcatcatggatggggtggaggtccctgaggagaatgtgctgcctggggctagctccctggggggcccctttggctgtctcaataatgctagatatggcattgcctggggggtgctgggggccagcgagttctgcctgcatacagctagacaatatgccctggacagaatgcagtttggggtgcccctggctagaaatcagctgattcagaagaagctggctgacatgctgacagagatcacactgggcctgcatgcctgtctgcagctggggagactgaaggaccaagataaggctgcccctgagatggtgagcctgctgaagagaaataactgtgggaaagctctggacattgctagacaagctagagacatgctggggggcaatggcatctccgatgagtaccatgtcatcagacatgccatgaacctggaggctgtgaacacctatgagggcacacatgacatccatgccctgatcctgggcagagccatcaccggcatccaagccttcacagctagcaagtgagaattccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 2226 bp, synthetic construct
  misc_feature 1..2226: note = V3
  source 1..2226: mol_type = other DNA
ctgcgcgctcgctcgctcactgaggccgcccgggcaaagcccgggcgtcgggcgacctttggtcgcccggcctcagtgagcgagcgagcgcgcagagagggagtggggttatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaattctcacttgctagctgtgaaggcttggatgccggtgatggctctgcccaggatcagggcatggatgtcatgtgtgccctcataggtgttcacagcctccaggttcatggcatgtctgatgacatggtactcatcggagatgccattgccccccagcatgtctctagcttgtctagcaatgtccagagctttcccacagttatttctcttcagcaggctcaccatctcaggggcagccttatcttggtccttcagtctccccagctgcagacaggcatgcaggcccagtgtgatctctgtcagcatgtcagccagcttcttctgaatcagctgatttctagccaggggcaccccaaactgcattctgtccagggcatattgtctagctgtatgcaggcagaactcgctggcccccagcaccccccaggcaatgccatatctagcattattgagacagccaaaggggccccccagggagctagccccaggcagcacattctcctcagggacctccaccccatccatgatgatcatcccggtggcggaagctctgaggctgaatttgccttggattctaggagcgctcaggcctctcatgcccttctccaggagaaagcctctgatacagccatcctcacatctagcccagaccacaaagagatcagccatggggctgtttgtgatccatgtcttggtgccattcagggtgtagctcttgttgctgctgttgtagtgggctcttgtctccatgctgctagggtcgctgccgctattgggctctgtgaggccaaagcagcccagcagctcccccttggccagctgagggagatatttctgtctctgctcctcgctgccataggcatagatggggtgcatgaccaggctgctctgcacgctcatagcgcttctgtacccgctgtccactctctccagctctctagccagcaggccataggccacgctgctcaccccagcacagccatagcccttgattgtagggcccagcacccccagctcccccatctcgctaatgatttctctgtggaagacctcatttctgttggccagcaggattctaggcatcagtctctcttggcagtaggttctgaaggtgtctctgatcaggatctcatctgtggtcagctgctcctccaggaccagggggtcttgccagtcaaattcaggtctgctgctcttggccagttggctctgtgttctgccccccttctctgtctgagcagcgctggacacccatgttctcagcacatgcaggccagggcctctgctcagcagtctgacgctcacccctctcagagccatggtggcggcaagcttctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgtcgcagcgactccctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcacccgttcaattgccgacccctccccccaacttctcggggactgtgggcgatgtgcgctctgcccactgacgggcaccggagccactagtcctaggtctagaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - DNA, 106 bp, synthetic construct
  misc_feature 1..106: note = L-LTR
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 23 - DNA, 130 bp, synthetic construct
  misc_feature 1..130: note = R-LTR
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 380 bp, synthetic construct
  misc_feature 1..380: note = CMV enhancer
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 25 - DNA, 204 bp, synthetic construct
  misc_feature 1..204: note = CMV promoter
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 26 - DNA, 278 bp, synthetic construct
  misc_feature 1..278: note = CBA promoter
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 27 - DNA, 91 bp, synthetic construct
  misc_feature 1..91: note = hGCDH intron 1
  source 1..91: mol_type = other DNA
gtcagtgtggggtcgggagtgtggagggaaggagggaggaactgggggtttagggactttccggggtgactttcccgttctgtgcttgcag

SEQ ID NO: 28 - DNA, 97 bp, synthetic construct
  misc_feature 1..97: note = SV40 intron
  source 1..97: mol_type = other DNA
gtaagtttagtctttttgtcttttatttcaggtcccggatccggtggtggtgcaaatcaaagaactgctcctcagtggatgttgcctttacttctag

SEQ ID NO: 29 - DNA, 231 bp, synthetic construct
  misc_feature 1..231: note = EF-1 core promoter
  source 1..231: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 30 - DNA, 308 bp, synthetic construct
  misc_feature 1..308: note = hGCDH intron 2
  source 1..308: mol_type = other DNA
gtaaggacctctggtcgcaccgtgtgtctgctgcccctgttcagctgtctgtctgccgcaggtggactctgtcccagaatccgagagctgcccgagcggggtggcagggtcgtggccagggtcagaggcactaaggcagtgagtgcgctgtgcctgcggggccggagaaaagtcacctgatcagtctcgcttgcagctcgcactagccggggggcgacatgggtgttggggggtagggctgatgagggtccgagaagggagggcacagtgatcttgcggactggaccgaggcgaattccccttcccag

SEQ ID NO: 31 - DNA, 141 bp, synthetic construct
  misc_feature 1..141: note = hGCDH intron 3
  source 1..141: mol_type = other DNA
gtgggcgggctggtgggtgccctgagactgctcctccgcctggagccatagccaccccacctcaaggcccctctgtccttggggctggggcttcctgtggcctaggcctgggcctgaatttgggcactggtccctttgcag

SEQ ID NO: 32 - DNA, 229 bp, synthetic construct
  misc_feature 1..229: note = hybrid intron
  source 1..229: mol_type = other DNA
ggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 33 - protein, 438 aa, synthetic construct
  REGION 1..438: note = GCDH prot
MALRGVSVRLLSRGPGLHVLRTWVSSAAQTEKGGRTQSQLAKSSRPEFDWQDPLVLEEQLTTDEILIRDTFRTYCQERLMPRILLANRNEVFHREIISEMGELGVLGPTIKGYGCAGVSSVAYGLLARELERVDSGYRSAMSVQSSLVMHPIYAYGSEEQRQKYLPQLAKGELLGCFGLTEPNSGSDPSSMETRAHYNSSNKSYTLNGTKTWITNSPMADLFVVWARCEDGCIRGFLLEKGMRGLSAPRIQGKFSLRASATGMIIMDGVEVPEENVLPGASSLGGPFGCLNNARYGIAWGVLGASEFCLHTARQYALDRMQFGVPLARNQLIQKKLADMLTEITLGLHACLQLGRLKDQDKAAPEMVSLLKRNNCGKALDIARQARDMLGGNGISDEYHVIRHAMNLEAVNTYEGTHDIHALILGRAITGIQAFTASK